SEQUENCE LISTING (45 sequences)

SEQ ID NO: 1 - DNA, 20 bp, Artificial Sequence
  Description of Artificial Sequence Synthetic oligonucleotide
ttgggccagagtacgatgca

SEQ ID NO: 2 - DNA, 20 bp, Artificial Sequence
  Description of Artificial Sequence Synthetic oligonucleotide
ctcatgaacggccaccaggt

SEQ ID NO: 3 - DNA, 20 bp, Artificial Sequence
  Description of Artificial Sequence Synthetic oligonucleotide
gggtcctggagctccatact

SEQ ID NO: 4 - DNA, 20 bp, Artificial Sequence
  Description of Artificial Sequence Synthetic oligonucleotide
tcatcctgttcggtcataat

SEQ ID NO: 5 - DNA, 20 bp, Artificial Sequence
  Description of Artificial Sequence Synthetic oligonucleotide
tagcatttgagctagttcga

SEQ ID NO: 6 - DNA, 20 bp, Artificial Sequence
  Description of Artificial Sequence Synthetic oligonucleotide
catgaagtcggcgctgacag

SEQ ID NO: 7 - DNA, 20 bp, Artificial Sequence
  Description of Artificial Sequence Synthetic oligonucleotide
ggtgcctccggacagcaaac

SEQ ID NO: 8 - DNA, 20 bp, Artificial Sequence
  Description of Artificial Sequence Synthetic oligonucleotide
aatggtgtctgcaagtggcc

SEQ ID NO: 9 - DNA, 20 bp, Artificial Sequence
  Description of Artificial Sequence Synthetic oligonucleotide
acccaggcatcatccgacaa

SEQ ID NO: 10 - DNA, 20 bp, Artificial Sequence
  Description of Artificial Sequence Synthetic oligonucleotide
tcatggctgggctctccagg

SEQ ID NO: 11 - DNA, 20 bp, Artificial Sequence
  Description of Artificial Sequence Synthetic oligonucleotide
atgggtctttctatcctatt

SEQ ID NO: 12 - DNA, 20 bp, Artificial Sequence
  Description of Artificial Sequence Synthetic oligonucleotide
tcagcagtttccctgctaat

SEQ ID NO: 13 - DNA, 20 bp, Artificial Sequence
  Description of Artificial Sequence Synthetic oligonucleotide
gatagaaagacccattagca

SEQ ID NO: 14 - DNA, 20 bp, Artificial Sequence
  Description of Artificial Sequence Synthetic oligonucleotide
gggtagtgagcgttgtacga

SEQ ID NO: 15 - DNA, 20 bp, Artificial Sequence
  Description of Artificial Sequence Synthetic oligonucleotide
ttctgcttcttcagcgggta

SEQ ID NO: 16 - DNA, 20 bp, Artificial Sequence
  Description of Artificial Sequence Synthetic oligonucleotide
tccagaatggagtcgcaggt

SEQ ID NO: 17 - DNA, 20 bp, Artificial Sequence
  Description of Artificial Sequence Synthetic oligonucleotide
gttgatgaaacagagcactt

SEQ ID NO: 18 - DNA, 20 bp, Artificial Sequence
  Description of Artificial Sequence Synthetic oligonucleotide
tgcctctctccggattgcca

SEQ ID NO: 19 - DNA, 20 bp, Artificial Sequence
  Description of Artificial Sequence Synthetic oligonucleotide
gttccttacttcagtctcta

SEQ ID NO: 20 - DNA, 20 bp, Artificial Sequence
  Description of Artificial Sequence Synthetic oligonucleotide
gcatctgtcacgtaagacag

SEQ ID NO: 21 - DNA, 20 bp, Artificial Sequence
  Description of Artificial Sequence Synthetic oligonucleotide
ctggattccactttccaacc

SEQ ID NO: 22 - DNA, 20 bp, Artificial Sequence
  Description of Artificial Sequence Synthetic oligonucleotide
gtgtgcgaccattgttcggg

SEQ ID NO: 23 - DNA, 20 bp, Artificial Sequence
  Description of Artificial Sequence Synthetic oligonucleotide
gacattggtacgggatttcc

SEQ ID NO: 24 - DNA, 20 bp, Artificial Sequence
  Description of Artificial Sequence Synthetic oligonucleotide
ctgatcgaccagatcgacag

SEQ ID NO: 25 - DNA, 20 bp, Artificial Sequence
  Description of Artificial Sequence Synthetic oligonucleotide
gtgtacaggattgttcctga

SEQ ID NO: 26 - DNA, 20 bp, Artificial Sequence
  Description of Artificial Sequence Synthetic oligonucleotide
ctggacggcggcaaagccct

SEQ ID NO: 27 - DNA, 20 bp, Artificial Sequence
  Description of Artificial Sequence Synthetic oligonucleotide
gagctgtactcgggcacgta

SEQ ID NO: 28 - DNA, 20 bp, Artificial Sequence
  Description of Artificial Sequence Synthetic oligonucleotide
gtactgcgccgcgtccatgt

SEQ ID NO: 29 - DNA, 40 bp, Unknown
  Description of Unknown FOXP3 sequence
tgatcatggctgggctctccagggggtgcacctgcagcac

SEQ ID NO: 30 - DNA, 40 bp, Unknown
  Description of Unknown FOXP3 sequence
tgatcatggctgggctctccagggggtgcacctgcaccac

SEQ ID NO: 31 - DNA, 40 bp, Unknown
  Description of Unknown FOXP3 sequence
tgatcatggctgggctctccagggggtgctcctgcagcac

SEQ ID NO: 32 - DNA, 40 bp, Unknown
  Description of Unknown FOXP3 sequence
tgatcatggctgggctccccagggggtgcacctgcagcac

SEQ ID NO: 33 - DNA, 40 bp, Unknown
  Description of Unknown FOXP3 sequence
atcatggctgggctctccacagggggtgcacctgcagcac

SEQ ID NO: 34 - DNA, 40 bp, Unknown
  Description of Unknown FOXP3 sequence
tgatcatggctgggctctccggggggtgcacctgcagcac

SEQ ID NO: 35 - DNA, 33 bp, Unknown
  Description of Unknown FOXP3 sequence
tgatcatggctggagggggtgcacctgcagcac

SEQ ID NO: 36 - DNA, 40 bp, Unknown
  Description of Unknown FOXP3 sequence
tgatcatggctgggctctcctagggggtgcacctgcagca

SEQ ID NO: 37 - DNA, 38 bp, Unknown
  Description of Unknown FOXP3 sequence
tgatcatggctgggctccagggggtgcacctgcagcac

SEQ ID NO: 38 - DNA, 21 bp, Unknown
  Description of Unknown FOXP3 sequence
tgatcatggcacctgcagcac

SEQ ID NO: 39 - DNA, 26 bp, Unknown
  Description of Unknown FOXP3 sequence
tgatcatggctgggcacctgcagcac

SEQ ID NO: 40 - DNA, 36 bp, Unknown
  Description of Unknown FOXP3 sequence
tgatcatggctgggccagggggtgcacctgcagcac

SEQ ID NO: 41 - DNA, 35 bp, Unknown
  Description of Unknown FOXP3 sequence
tgatcatggctgggcagggggtgcacctgcagcac

SEQ ID NO: 42 - DNA, 40 bp, Unknown
  Description of Unknown FOXP3 sequence
gatcatggctgggctctcccagggggtgcacctgcagcac

SEQ ID NO: 43 - DNA, 18 bp, Unknown
  Description of Unknown FOXP3 sequence
tgatcatggctgcagcac

SEQ ID NO: 44 - DNA, 39 bp, Unknown
  Description of Unknown FOXP3 sequence
tgatcatggctgggctctccgggggtgcacctgcagcac

SEQ ID NO: 45 - DNA, 39 bp, Unknown
  Description of Unknown FOXP3 sequence
tgatcatggctgggctctcagggggtgcacctgcagcac

(19) United States
(12) Patent Application Publication
(10) Pub. No.: US 2021/0147841 A1
(43) Pub. Date: May 20, 2021

(54) COMPOSITIONS AND METHODS FOR MODIFYING REGULATORY T CELLS

(71) Applicant: The Regents of the University of California, Oakland, CA (US)

(72) Inventors: Alexander Marson, San Francisco, CA (US); Jeffrey A. Bluestone, San Francisco, CA (US); Kathrin Schumann, Munich (DE)

(21) Appl. No.: 17/089,284

(22) Filed: Nov. 4, 2020

Related U.S. Application Data
(63) Continuation of application No. PCT/US2019/031119, filed on May 7, 2019.
(60) Provisional application No. 62/667,981, filed on May 7, 2018.

(51) Int. Cl. C12N 15/113; C12N 9/22; C12N 5/0783

(52) U.S. Cl. CPC C12N 15/113; C12N 9/22; C12N 2510/00; C12N 2310/20; C12N 5/0637

(57) ABSTRACT

The disclosure features methods directed to modifying regulatory T (Treg) cell stability by inhibiting the expression of one or more transcription factors and/or inhibiting one or more genes or gene products regulated by the transcription factors. The disclosure also features compositions comprising the Treg cells having modified stability.

### CROSS-REFERENCES TO RELATED APPLICATIONS

The present application is a continuation of International Application No. PCT/US2019/031119, filed May 7, 2019, which claims priority to U.S. Provisional Patent Application No. 62/667,981 filed, May 7, 2018, the contents of each of which are incorporated herein by reference in their entirety.

### REFERENCE TO A “SEQUENCE LISTING,” A TABLE, OR A COMPUTER PROGRAM LISTING APPENDIX SUBMITTED ON A COMPACT DISK

This application is being filed electronically and includes an electronically submitted sequence listing in .txt format. The .txt file contains a sequence listing entitled “081906-1213548-229320US_SL.txt” created on Nov. 3, 2020 and having a size of 10,331 bytes. The sequence listing contained in this .txt file is part of the specification and is herein incorporated by reference in its entirety.

### BACKGROUND OF THE DISCLOSURE

Regulatory T cells (Treg cells) play a role in regulating immune response. In some cases, for example in some cancers, Treg cells inhibit the ability of the immune system to target and destroy cancer cells. In other cases, for example in autoimmune diseases, Treg cells are unavailable to control the immune system.

### BRIEF SUMMARY OF THE DISCLOSURE

In one aspect, the disclosure features a method of modifying regulatory T (Treg) cell stability, the method comprising: inhibiting expression of one or more transcription factors (TFs) selected from the group consisting of CIC, FOXO1, FOXP3, IKZF2, PRDM1, HIVEP2, SATB1, IRF4, T-BET, and GATA3 and/or inhibiting expression of one or more genes or gene products regulated by one or more of the transcription factors in the Treg cell.

In some embodiments, the inhibiting the expression destabilizes the Treg cell. In other embodiments, the inhibiting the expression stabilizes the Treg cell. In some embodiments, the method comprises inhibiting expression of one or more transcription factors selected from the group consisting of CIC, FOXO1, FOXP3, IKZF2, PRDM1, HIVEP2, SATB1, IRF4, T-bet, and GATA3 (e.g., FOXP3, IRF4, FOXO1, PRDM1, SATB1, and HIVEP2). In particular embodiments, the transcription factors are FOXO1 and IRF4. In particular embodiments, the transcription factors are HIVEP2 and SATB1. In some embodiments, the method comprises inhibiting expression of one or more genes or gene products regulated by one or more of the transcription factors.

In some embodiments, the inhibiting comprises reducing expression of the transcription factor, reducing expression of a polynucleotide encoding the transcription factor, or reducing expression of the gene or gene product regulated by the transcription factor.

In some embodiments, the inhibiting comprises contacting a polynucleotide encoding the transcription factor or a polynucleotide encoding the gene or gene product regulated by the transcription factor with a target nuclease, a guide RNA (gRNA), an siRNA, an antisense RNA, microRNA (miRNA), or short hairpin RNA (shRNA).

In some embodiments, the inhibiting comprises contacting a polynucleotide encoding the transcription factor or a polynucleotide encoding the gene or gene product regulated by the transcription factor with at least one gRNA (and optionally a targeted nuclease), wherein the at least one gRNA comprise a sequence selected from Table 2.

In other embodiments, the inhibiting comprises mutating a polynucleotide encoding the transcription factor or a polynucleotide encoding the gene regulated by the transcription factor. In certain embodiments, the inhibiting comprises contacting the polynucleotide with a targeted nuclease. The targeted nuclease may introduce a double-stranded break in a target region in the polynucleotide. The targeted nuclease may be an RNA-guided nuclease. In some embodiments, the RNA-guided nuclease is a Cpf1 nuclease or a Cas9 nuclease and the method further comprises introducing into the Treg cell a gRNA that specifically hybridizes to the target region in the polynucleotide.

In some embodiments, the Cpf1 nuclease or the Cas9 nuclease and the gRNA are introduced into the cell as a ribonucleoprotein (RNP) complex.

In some embodiments, the inhibiting comprises performing clustered regularly interspaced short palindromic repeats (CRISPR)/Cas genome editing.

In other embodiments, the Treg cell is administered to a human following the inhibiting.

In some embodiments of this aspect, the Treg cell is obtained from a human, the Treg cell so obtained is treated to inhibit the expression, and then the cell having inhibited expression is reintroduced to the human. In certain embodiments, the inhibiting the expression results in the Treg cell with increased stability. Treg cells with increased stability may be used to treat an autoimmune disease in a human. In certain embodiments, the inhibiting the expression results in the Treg cell with decreased stability. Treg cells with decreased stability may be used to treat cancer in a human.

In another aspect, the disclosure features a Treg cell made by any of the methods described herein.

In another aspect, the disclosure features a Treg cell comprising a genetic modification or heterologous polynucleotide that inhibits expression of one or more transcription factors selected from the group consisting of CIC, FOXO1, FOXP3, IKZF2, PRDM1, HIVEP2, SATB1, IRF4, T-bet, and GATA3 (e.g., FOXP3, IRF4, FOXO1, PRDM1, SATB1, and HIVEP2) and/or inhibits expression of one or more genes or gene products regulated by one or more of the transcription factors in the Treg cell. In particular embodiments, the transcription factors are FOXO1 and IRF4. In particular embodiments, the transcription factors are HIVEP2 and SATB1.

In another aspect, the disclosure features a Treg cell comprising at least one guide RNA (gRNA) comprising a sequence selected from Table 2. In some embodiments, the expression of one or more transcription factors in the Treg cell (e.g., CIC, FOXO1, FOXP3, IKZF2, PRDM1, HIVEP2, SATB1, IRF4, T-bet, and GATA3 (e.g., FOXP3, IRF4, FOXO1, PRDM1, SATB1, and HIVEP2)) and/or the expression of one or more genes or gene products regulated by the one or more of the transcription factors is reduced in the Treg cell relative to the expression of the transcription factor, gene, or gene product in a Treg cell not comprising a gRNA. In particular embodiments, the transcription factors are FOXO1 and IRF4. In particular embodiments, the transcription factors are HIVEP2 and SATB1.

### Definitions

As used in this specification and the appended claims, the singular forms “a,” “an,” and “the” include plural reference unless the context clearly dictates otherwise.

The term “nucleic acid” or “nucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a particular nucleic acid sequence may include degenerate codon substitutions, alleles, orthologs, SNPs, and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” can refer to the segment of DNA involved in producing or encoding a polypeptide chain. It may include regions preceding and following the coding region (leader and trailer) as well as intervening sequences (introns) between individual coding segments (exons).

The term “inhibiting expression” refers to inhibiting or reducing the expression of a gene or a protein. To inhibit or reduce the expression of a gene (i.e., a gene encoding a transcription factor, or a gene regulated by a transcription factor), the sequence and/or structure of the gene may be modified such that the gene would not be transcribed (for DNA) or translated (for RNA), or would not be transcribe or translated to produce a functional protein (e.g., a transcription factor). Various methods for inhibiting or reducing expression of a gene are described in detail further herein. Some methods may introduce nucleic acid substitutions, additions, and/or deletions into the wild-type gene. Some methods may also introduce single or double strand breaks into the gene. To inhibit or reduce the expression of a protein (e.g., a transcription factor), one may inhibit or reduce the expression of the gene or polynucleotide encoding the protein, as described above. In other embodiments, one may target the protein directly to inhibit or reduce the protein's expression using, e.g., an antibody or a protease.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequences that are perfectly complementary or substantially complementary (e.g., having 1-4 mismatches) to a genomic sequence.

As used throughout, by subject is meant an individual. For example, the subject is a mammal, such as a primate, and, more specifically, a human. The term does not denote a particular age or sex. Thus, adult and newborn subjects, whether male or female, are intended to be covered. As used herein, patient or subject may be used interchangeably and can refer to a subject afflicted with a disease or disorder.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. Variants of any of the Cas9 nucleases provided herein can be optimized for efficient activity or enhanced stability in the host cell. Thus, engineered Cas9 nucleases are also contemplated.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the targeting sequence may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence. Table 2 shows exemplary gRNA sequences used in methods of the disclosure.

As used herein, the term “Cas9” refers to an RNA-mediated nuclease (e.g., of bacterial or archeal orgin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p 759-771, 22 Oct. 2015) and homologs thereof. Similarly, as used herein, the term “Cas9 ribonucleoprotein” complex and the like refers to a complex between the Cas9 protein and a guide RNA, the Cas9 protein and a crRNA, the Cas9 protein and a trans-activating crRNA (tracrRNA), or a combination thereof (e.g., a complex containing the Cas9 protein, a tracrRNA, and a crRNA guide RNA). It is understood that in any of the embodiments described herein, a Cas9 nuclease can be substituted with a Cpf1 nuclease or any other guided nuclease.

As used herein, the phrase “modifying” in the context of modifying a genome of a cell refers to inducing a structural change in the sequence of the genome at a target genomic region. For example, the modifying can take the form of inserting a nucleotide sequence into the genome of the cell. For example, a nucleotide sequence encoding a polypeptide can be inserted into the genomic sequence encoding an endogenous cell surface protein in the T cell. The nucleotide sequence can encode a functional domain or a functional fragment thereof. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region.

As used herein, the phrase “introducing” in the context of introducing a nucleic acid or a complex comprising a nucleic acid, for example, an RNP complex, refers to the translocation of the nucleic acid sequence or the RNP complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1: Deregulation of several Treg/Teff markers in FOXP3 knocked-out Treg cells.

FIG. 2: Heatmap summarizing the results of FOXP3 screen for 39 transcription factors.

FIG. 3: Heatmap summarizing the results of CTLA4 screen for 39 transcription factors.

FIG. 4: Heatmap summarizing the results of IFNγ screen for 39 transcription factors.

FIGS. 5 and 6: Comparison of the results of pooled (log 2 fold change editing efficiencies in exTreg/Treg fraction) versus arrayed (% protein expression changes based on FACS) screens.

FIGS. 7A-7J: Multidimensional FACS analysis of 10 transcription factors.

FIG. 8: Clustering of single cell RNA-sequencing (scRNA-seq) data of 10 transcription factors.

FIGS. 9A-9F: Pooled Cas9 RNP screens identify regulators of FOXP3, CTLA-4 and IFNg gene expression in a cytokine-dependent manner. FIG. 9A: Schematic workflow of pooled Cas9 RNP screens. FIG. 9B: Sorting strategy to isolate IFNg-positive and -negative Tregs in ctrl (top) and in with pool of RNP targeting 40 individual TFs edited Tregs (bottom). Ctrl and “Pool RNP” Tregs were stimulated with IL-2 only (w/o) or IL-2 and IL-4, IL-6, IL-12 or IFNg. FIG. 9C: Examples of indels found at the FOXP3 target locus in sorted FOXP3+ and FOXP3− cell populations. FIG. 9D: log 2 fold enrichment of indels in FOXP3+/FOXP3− cell populations. On the left with FOXP3 included (positive control), on the right extended view on residual TFs. FIG. 9E: log 2 fold enrichment of indels in CTLA-4+/CTLA-4− cell populations. FIG. 9F: log 2 fold enrichment of indels in IFNg+/IFNg− cell populations. D-F: Mean of log 2 fold enrichment of 4 experiments in 4 donors.

FIGS. 10A-10C: Arrayed RNP screens for comprehensive phenotyping of TF KO Tregs. FIG. 10A: Workflow of arrayed Cas9 RNP screens to identify TFs regulating Treg identity. FACS panel of canonical Treg and effector T cell protein markers used as readout is shown on the left. FIG. 10B: PCA-plot summarizing FACS results of 9 protein markers of Cas9 RNP arrayed screens targeting 40 TFs without and with IL-12 cytokine challenge in 2 donors. Mean of 3 independent gRNAs targeting one TF for each individual condition are shown. FIG. 10C: Comparison of results generated in pooled (log(indels in IFNg high population/indels in IFNg low population) versus arrayed CRISPR RNP screens (log(% IFNg positive in KO/% IFNg positive in ctrl)) selecting on IFNg secretion with and without IL-12 conditioning. Grey zone: confidence interval.

FIGS. 11A and 11B: In depth phenotyping of loss of Treg cell identity in CTRL, IKZF2 KO and FOXP3 KO Tregs. FIG. 11A: Left: Representative FACS results for CTRL, IKZF2 KO and FOXP3 KO Tregs without IL-12 stimulation. Tregs were targeted with one representative RNP in one Donor. Right: Personality plots summarizing the FACS results for all 9 markers. Each dimension—representing a FACS marker—of a given personality plot represents the ratio of the percent positive TF KO cells for that marker to the average of percent positive control cells for the respective marker. Red lines indicate the average over all control cells shown (6 controls). Black lines indicate the % difference to the average of the controls. FIG. 11B: FACS results and personality plots for CTRL, IKZF2 KO and FOXP3 KO Tregs with IL-12 conditioning (same RNPs and same donor as in A). For FOXP3 personality plots IL-4 was excluded because of the high fold change which made the visualization of the other FACS markers impossible.

FIGS. 12A-12D: 9-dimensional analysis of FACS data via SCAFFOLD to identify sub-phenotypes in TF KO conditions. FIG. 12A: SCAFFOLD plots of CTRL cells (representative example) with and without IL-12 conditioning. Landmark nodes are labeled based on reference gates in control samples. FIGS. 12B and 12C: Representative SCAFFOLD plots of IKZF2 and FOXP3 KOs without cytokine challenge (blue) and after IL-12 conditioning (red). FIG. 12D: SCAFFOLD and personality plots of 10 TFs chosen for in depth analysis with scRNA-seq. Grey labels: Landmark nodes of interest.

FIGS. 13A-13D: scRNA-seq data reveal Treg cell deregulation induced by IL-12 stimulation and/or TF KO. FIG. 13A: TSNE-plot of scRNA-seq data of 10 TF KOs and control cells with and without IL-12 stimulation. 8 clusters could be identified. FIG. 13B: Density plots of CTRL, FOXP3 KO, SATB1 KO and HIVEP2 KO cells with and without IL-12 stimulation. FIG. 13C: Distribution of KO conditions without (blue) and with IL-12 stimulation (red) normalized to control cells with the corresponding cytokine treatment. FIG. 13D: Top 10 upregulated genes for each cluster mapped for each cell analyzed.

FIGS. 14A and 14B: Individual TFs control distinct gene modules regulating Treg cell identity. FIG. 14A: Force-directed graph of gene modules regulated by Treg TFs (yellow). Gene upregulation is indicated by green arrow, gene downregulation by red arrow. In blue: Cytokines regulated by the individual TFs. In orange: Regulation of TFs and chromatin modifiers by targeted TFs. FIG. 14B: Heatmap of differentially regulated genes shown in FIG. 14A.

### DETAILED DESCRIPTION OF THE DISCLOSURE

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Introduction

Regulatory T (Treg) cells with pro-inflammatory features have been described in different context and with a variety of phenotypes. For example, murine Treg cells may down-regulate their master transcription factor FOXP3. In humans and mice with autoimmune conditions, Treg cells with changed cytokine profile could be detected. However, the processes that lead to these heterogeneous phenotypes are incompletely understood. Methods to stabilize Treg cells for the treatment of autoimmune diseases or actively destabilize Treg cells to ablate tolerogenic effects in a tumor microenvironment have great therapeutic potential. However, to identify the potential targets in Treg cells that may affect their stability, a greater understanding of the transcriptional regulation in Treg cells that lead to a stabilized cell phenotype or a destabilized cell phenotype and how these transcriptional networks are affected by pro-inflammatory conditions is needed.

For example, FOXP3, the Treg master transcription factor, is essential for Treg development and maintenance of the Treg transcriptional landscape. Mutations or lack of FOXP3 lead to immunodysregulation, polyendocrinopathy, enteropathy, X-linked syndrome (IPEX) in humans, which is characterized by loss of immune tolerance and the development of a spectrum of autoimmune disorders (Brunkow et al., 2001). However, FOXP3 alone is not sufficient to shape the Treg transcriptional landscape. Transduction of T effector cells with FOXP3 or FOXP3 induction in naïve T cells by TGFβ cannot recapitulate the full Treg gene signature (Sugimoto et al., 2006; Hill et al., 2007). Furthermore, Treg-like cells have been described in IPEX patients. These cells express non-functional FOXP3 and lack suppressive capacity, yet can still possess aspects of the Treg gene signature (Gavin et al., 2007; Lin et al., 2007; Otsubo et al., 2011). In Type 1 Diabetes and multiple sclerosis, FOXP3+ IFNγ+ cells have been characterized (Dominguez-Villar et al., 2011; McClymont et al., 2011). In Crohn's disease, FOXP3-expressing Tregs that also secrete IL-17a have been identified (Hovhannisyan et al., 2011). These observations support the idea that Tregs can have a variety of phenotypes, and FOXP3 expression alone does not fully control Treg transcriptional identity.

In mice, several FOXP3 interacting TFs including Gata3 (Rudra et al., 2012) and Foxp1 (Konopacki et al., 2019) have been described that reinforce the Treg gene signature. A quintet of transcription factors, SATB1, IKZF4, IRF4, LEF-1, and GATA-1, were identified that synergize with FOXP3 to “lock in” the expression of signature Treg genes in mature Tregs. These TFs synergize with FOXP3 to induce the expression of signature Treg genes, including downstream TFs, and act as FOXP3 co-factors to reinforce FOXP3 binding to regulatory DNA sequences (Fu et al., 2012). However, the understanding around the key TFs regulating human Treg cell identity is still scarce.

Cytokines and other extracellular clues interact with Treg TFs to influence the Treg transcriptional landscape. IL-6, a pro-inflammatory cytokine that promotes Th17 cell generation is one such factor and can down-regulate FOXP3 and the suppressive capacities of Tregs in vitro (Pasare et al., 2003). High IFNγ levels in the tumor microenvironment negatively can affect Treg cells' anti-inflammatory features and turns these cells into IFNγ producers themselves (Xu et al., 2017). In viral-induced inflammatory lesions, Tregs can lose FOXP3 expression adopt a Th1-like phenotype. The Th1-Treg state appears to be at least partly dependent on the exposure to IL-12 (Bhela et al., 2017). These data indicate that an interplay of cell extrinsic and intrinsic factors affect Treg identity.

Most of the functional knowledge of Treg gene regulation is derived from mouse models. The development of CRISPR Cas9 RNP technology allows the dissection of genetic modules in primary human Tregs with targeted gene perturbation studies. Here, a set of TFs that control critical gene targets in human Tregs were identified. CRISPR perturbation with scRNA-seq revealed genetic modules that are controlled by these TF directly or indirectly. Further, how these transcriptional regulatory networks depend upon both lineage TFs and an extracellular cytokine environment was characterized.

Forty candidate TFs that were preferentially expressed in Tregs compared to other effector T cell subsets or have been indicated in regulating cell identity of murine Tregs were first analyzed. These TFs were ablated in pooled and arrayed Cas9 RNP screens under different pro-inflammatory conditions. The combined effects of genetic perturbation and microenvironment on canonical Treg and T effector proteins were measured via FACS. Pro-inflammatory cytokine challenge enhanced a multitude of knock-out (KO) phenotypes, revealing cytokine-responsive genes that are normally repressed by Treg TFs. Based on these results, 10 TFs were selected for in depth analysis by scRNA-seq. By combining TF KO, cytokine stimulation, and scRNA-seq, gene modules regulated by distinct TFs were identified. These gene modules give insights into the transcriptional regulation of cytokines, co-inhibitory receptors, and TFs in human Tregs.

As described herein, distinct gene modules are regulated by FOXP3 and PRDM1. IRF4 and FOXO1 co-regulate a large gene subset, but also individually control distinct gene modules. HIVEP2 has not been previously characterized in Tregs. As described herein, a large set of genes in human Tregs depends on both HIVEP2 and SATB1 for proper gene activation, particularly in the presence of pro-inflammatory IL12. The functional gene perturbation studies provide a powerful single-cell resolution resource to inform future development of drug targets and design of Treg-based cell therapies to treat immune dysregulation and cancer.

In some embodiments, CRISPR/Cas9 genome editing may be used to target and modify transcription factors in human Treg cells in order to study the influence of certain transcription factors on Treg cell stability and maintenance. Furthermore, the loss of transcription factors may affect individual cells differently due to stochastic effects within the cells. Single-cell resolution of the different genetic knock-outs may provide the opportunity to distinguish different subpopulations of destabilized or stabilized Treg cells with distinct acquired effector functions.

As described herein, the disclosure provides compositions and methods directed to modifying regulatory T (Treg) cell stability by inhibiting the expression of one or more transcription factors (e.g., CIC, FOXO1, FOXP3, IKZF2, PRDM1, HIVEP2, SATB1, IRF4, T-BET, and GATA3) and/or inhibiting one or more genes or gene products regulated by the transcription factors. The disclosure also features compositions comprising the Treg cells having modified stability. A population of modified Treg cells that are destabilized may be provide therapeutic benefits in treating cancer. A population of modified Treg cells that are stabilized may provide therapeutic benefits in treating autoimmune diseases.

### II. Methods and Compositions

The present disclosure is directed to compositions and methods for modifying the stability of regulatory T cells (also referred to as “Treg cells”). The inventors have discovered that by inhibiting the expression of one or more transcription factors and/or inhibiting the expression of one or more genes regulated by the one or more transcription factors, the stability of Treg cells may be altered. In some embodiments, the Treg cells may be destabilized by inhibiting the expression of one or more transcription factors and/or inhibiting the expression of one or more genes regulated by the transcription factors, such that they may have less immunosuppressive effects and improved therapeutic benefits towards treating cancer. A population of destabilized Treg cells may be used to enhance or improve various cancer therapies or Treg cells of an individual having cancer can be targeted to destabilize the Treg cells. In other embodiments, the stability of the Treg cells may be improved by inhibiting the expression of one or more transcription factors and/or inhibiting the expression of one or more genes regulated by the transcription factors, such that they may have more immunosuppressive effects. A population of stabilized Treg cells may be used to treat or alleviate autoimmune diseases. Examples of transcription factors whose expression may be altered to modify the stability of Treg cells in the methods described herein include, but are not limited to, BACH1, CIC, ELF1, FOXO1, FOXO4, FOXP1, FOXP3, HIVEP1, ID3, IKZF2, IKZF4, MXD1, NCOR1, NR4A1, PRDM2, SP4, TGIF1, ZBTB38, ZC3H7A, ZEB1, ZFY, ZNF335, ZNF480, ZNF532, ZNF831, TCF7, PRDM1, JAZF1, HIVEP2, SATB1, GATA1, IRF4, LEF1, XBP1, NFATC2, BACH2, FOXO3, EOMES, T-BET, and GATA3. In particular embodiments, examples of transcription factors whose expression may be altered to modify the stability of Treg cells in the methods described herein include CIC, FOXO1, FOXP3, IKZF2, PRDM1, HIVEP2, SATB1, IRF4, T-BET, and GATA3.

In some embodiments, stability of the Treg cells may indicate a state of Treg cells as they undergo modifications that inhibit the expression of one or more transcription factors in the cells and/or inhibit the expression of one or more genes or gene products regulated by the transcription factors. Stability of the Treg cells may be assessed using data from the arrayed screen and the FACS readout (9 FACS markers). Some of the FACS markers used are canonical Treg cell signature proteins. Some of the FACS markers are proteins that are normally not expressed in Treg cells, but are expressed under pro-inflammatory challenges. Using the FACS markers, the loss of Treg cell canonical markers and/or gain of pro-inflammatory markers were assessed and analyzed to determine the change in Treg cell stability. For selected transcription factors, single cell transcriptome changes were also assessed in Treg cells treated with Cas9 RNP complex, which provides more information regarding how Treg cell states are changed as a result of the loss of certain transcription factors. For both flow and single cell transcriptome analysis, Treg cell state was assessed in the presence and absence of IL-12, a pro-inflammatory cytokine. This was done to assess the role of certain transcription factors in guarding against destabilization in the face of pro-inflammatory stimulus. For example, with a specific transcription factor knocked-out in Treg cells, if these modified cells display a gain or maintenance of Treg cell canonical markers, such as FOXP3, CTLA4, CD25, IL-10, and IKZF2, then the transcription factor is likely not to have guarding effects against destabilization. In some embodiments, a loss of Treg cell canonical markers and/or gain of pro-inflammatory markers (e.g., IL-17a, IL-4, IFNγ, and IL-2) may indicate that the Treg cells are destabilized. In some embodiments, a gain or maintenance of Treg cell canonical markers, such as FOXP3, CTLA4, CD25, IL-10, and IKZF2, may indicate that the Treg cells are more stabilized.

In some embodiments of the methods described herein, inhibiting the expression of the one or more transcription factors and/or inhibiting the expression of one or more genes regulated by the transcription factors may comprise reducing expression of the transcription factor, reducing expression of a polynucleotide encoding the transcription factor, and/or reducing expression of the gene regulated by the transcription factor. As described in detail further herein, one or more available methods may be used to inhibit the expression of one or more transcription factors and/or to inhibit the expression of one or more genes regulated by the transcription factors.

In some embodiments, inhibiting the expression may comprise contacting a polynucleotide encoding the transcription factor or a polynucleotide encoding the gene regulated by the transcription factor with a target nuclease, a guide RNA (gRNA), an siRNA, an antisense RNA, microRNA (miRNA), or short hairpin RNA (shRNA). In particular embodiments, if a gRNA and a target nuclease (e.g., Cas9) are used to inhibit the expression of a polynucleotide encoding a transcription factor (e.g., a transcription factor selected from CIC, FOXO1, FOXP3, IKZF2, PRDM1, HIVEP2, SATB1, IRF4, T-BET, or GATA3), the gRNA may comprise a sequence selected from Table 2, or a portion thereof.

As described herein, the stability of Treg cells may be modified by inhibiting the expression of the one or more transcription factors (e.g., CIC, FOXO1, FOXP3, IKZF2, PRDM1, HIVEP2, SATB1, IRF4, T-BET, or GATA3) and/or one or more genes regulated by the transcription factors. Subsequently, once modified Treg cells are created, the modified Treg cells may be administered to a human. Depending on whether the Treg cells are stabilized or destabilized, the modified Treg cells may be used to treat different indications. For example, Treg cells may be isolated from a whole blood sample of a human and expanded ex vivo. The expanded Treg cells may then be treated to inhibit the expression of certain transcription factors or certain genes regulated by the transcription factors, thus, creating modified Treg cells. The modified Treg cells may be reintroduced to the human to treat certain indications. In some embodiments, destabilized Treg cells having less immunosuppressive effects may be used to treat cancer. In some embodiments, stabilized Treg cells having improved immunosuppressive effects may be used to treat autoimmune diseases. As shown in Table 1 below, certain transcription factors in Treg cells have a destabilizing effect once their expression is inhibited, while other transcription factors in Treg cells have a stabilizing effect once their expression is inhibited. Cell stability was determined by a multi-color FACS panel based on Treg cell markers like Foxp3, Helios, CTLA-4, CD25, IL-10, and effectors such as cytokines typically associated with effector T cell subsets like IL-2, IFNγ, IL-17a, and IL-4. Depending on the indication and the therapeutic needs, one may choose to target specific transcription factors to generate achieve modified Treg cells that are destabilized or stabilized.

TABLE 1
(inc: increase; decr: decrease; ind: induction)

| | | | Treg cell marker used in FACS | | | | | | | | |
|---|---|---|---|---|---|---|---|---|---|---|---|
| Transcription | | | | | | | | | Helios | | |
| Factor | Stimulation | Effect | FOXP3 | CD25 | CTLA4 | IFNg | IL-17a | IL-4 | (IKZF2) | IL-2 | IL-10 |
| FOXP3 | None | destabilizing | decr | decr | decr | inc | inc | inc | | inc | |
| | | | (control) | | | | | | | | |
| IRF4 | None | destabilizing | | | decr | inc | decr | inc | inc | inc | inc |
| PRDM1 | None | destabilizing | decr | | decr | | | | inc | inc | inc |
| FOXO1 | None | destabilizing | | | decr | inc | | | inc | | |
| IKZF2 | None | destabilizing | | | | | decr | | decr | | |
| | | | | | | | | | (control) | | |
| TBX21 | None | stabilizing | | | | decr | | | | | |
| | | | | | | (control) | | | | | |
| GATA3 | None | destabilizing | decr | | decr | | | decr | | | |
| SATB1 | None | stabilizing | | | ind | | | | | | |
| FOXP3 | IL-12 | destabilizing | decr | decr | decr | inc | inc | inc | | inc | |
| | | | (control) | | | (stronger | (less | | | | |
| | | | | | | than w/o | than w/o | | | | |
| | | | | | | IL-12) | IL-12) | | | | |
| IRF4 | IL-12 | destabilizing | | decr | decr | inc | | inc | inc | inc | inc |
| | | | | | | (stronger | | | | | |
| | | | | | | than w/o | | | | | |
| | | | | | | IL-12) | | | | | |
| PRDM1 | IL-12 | destabilizing | decr | | decr | | | | inc | inc | inc |
| FOXO1 | IL-12 | destabilizing | | decr | decr | inc | | | inc | decr | |
| | | | | | | (stronger | | | | | |
| | | | | | | than w/o | | | | | |
| | | | | | | IL-12) | | | | | |
| IKZF2 | IL-12 | destabilizing | | | | decr | | | decr | | |
| | | | | | | (control) | | | (control) | | |
| TBX21 | IL-12 | control | | | | | | | | | |
| GATA3 | IL-12 | destabilizing | decr | | decr | decr | | | | | |
| SATB1 | IL-12 | stabilizing | | | ind | | | | | | |

Furthermore, the disclosure also features a Treg cell comprising a genetic modification or heterologous polynucleotide that inhibits expression of one or more transcription factors (e.g., CIC, FOXO1, FOXP3, IKZF2, PRDM1, HIVEP2, SATB1, IRF4, T-bet, and GATA3) and/or inhibits expression of one or more genes regulated by one or more of the transcription factors in the Treg cell. A genetic modification may be a nucleotide mutation or any sequence alteration in the polynucleotide encoding the transcription factor that results in the inhibition of the expression of the transcription factor. A genetic modification may also be a nucleotide mutation or any sequence alteration in a gene regulated by the transcription factor. A heterologous polynucleotide may refer to a polynucleotide originally encoding the transcription factor but is altered, i.e., comprising one or more nucleotide mutations or sequence alterations. A heterologous polynucleotide may also refer to a polynucleotide originally encoding the gene regulated by the transcription factor, but is altered, i.e., comprising one or more nucleotide mutations or sequence alterations. Also disclosed herein are Treg cells comprising at least one guide RNA (gRNA) comprising a sequence selected from Table 2. The expression of one or more transcription factors (e.g., CIC, FOXO1, FOXP3, IKZF2, PRDM1, HIVEP2, SATB1, IRF4, T-bet, and GATA3) in the Treg cells comprising the gRNAs may be reduced in the Treg cells relative to the expression of the transcription factor in Treg cells not comprising the gRNAs. In some embodiments, the expression of one or more genes regulated by a transcription factor (e.g., CIC, FOXO1, FOXP3, IKZF2, PRDM1, HIVEP2, SATB1, IRF4, T-bet, or GATA3) in the Treg cells comprising the gRNAs may be reduced in the Treg cells relative to the expression of the gene in Treg cells not comprising the gRNAs.

In some embodiments, the RNP complex may be introduced into about 1×10⁵ to about 2×10⁶ cells (e.g., 1×10⁵ cells to about 5×10⁵ cells, about 1×10⁵ cells to about 1×10⁶ cells, 1×10⁵ cells to about 1.5×10⁶ cells, 1×10⁵ cells to about 2×10⁶ cells, about 1×10⁶ cells to about 1.5×10⁶ cells, or about 1×10⁶ cells to about 2×10⁶ cells). In some embodiments, the Treg cells are cultured under conditions effective for expanding the population of modified Treg cells. Also disclosed herein is a population of Treg cells, in which the genome of at least 20%, 30%, 40%, 50%, 60%, 70%, 80%, 90%, 95%, 99% or greater of the cells comprises a genetic modification or heterologous polynucleotide that inhibits expression of one or more transcription factors (e.g., CIC, FOXO1, FOXP3, IKZF2, PRDM1, HIVEP2, SATB1, IRF4, T-bet, and GATA3) and/or inhibits expression of one or more genes regulated by one or more of the transcription factors in the Treg cell.

In some embodiments, the RNP complex is introduced into the Treg cells by electroporation. Methods, compositions, and devices for electroporating cells to introduce a RNP complex are available in the art, see, e.g., WO 2016/123578, WO/2006/001614, and Kim, J. A. et al. Biosens. Bioelectron. 23, 1353-1360 (2008). Additional or alternative methods, compositions, and devices for electroporating cells to introduce a RNP complex can include those described in U.S. Patent Appl. Pub. Nos. 2006/0094095; 2005/0064596; or 2006/0087522; Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842; Geng, T. et al., J. Control Release 144, 91-100 (2010); and Wang, J., et al. Lab. Chip 10, 2057-2061 (2010).

### III. Methods of Inhibiting Expression

CRISPR/Cas Genome Editing

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “guide RNA” or “gRNA”), and the crRNA equivalent portion of the single guide RNA can be engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments of the methods described herein, CRISPR/Cas genome editing may be used to inhibit the expression of one or more transcription factors selected from the group consisting of CIC, FOXO1, FOXP3, IKZF2, PRDM1, HIVEP2, SATB1, IRF4, T-bet, and GATA3. In other embodiments, CRISPR/Cas genome editing may be used to knock out one or more genes regulated by one or more transcription factors selected from the group consisting of CIC, FOXO1, FOXP3, IKZF2, PRDM1, HIVEP2, SATB1, IRF4, T-bet, and GATA3.

In some embodiments, the Cas nuclease has DNA cleavage activity. The Cas nuclease can direct cleavage of one or both strands at a location in a target DNA sequence, i.e., a location in a polynucleotide encoding a transcription factor selected from the group consisting of CIC, FOXO1, FOXP3, IKZF2, PRDM1, HIVEP2, SATB1, IRF4, T-bet, and GATA3. In some embodiments, the Cas nuclease can be a nickase having one or more inactivated catalytic domains that cleaves a single strand of a target DNA sequence.

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP 269215, and the amino acid sequence of Streptococcus thermophilus wild-type Cas9 polypeptide is set forth, e.g., in NBCI Ref. Seq. No. WP_011681470. Some CRISPR-related endonucleases that may be used in methods described herein are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 may be a fusion protein, e.g., the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the Cas9 nuclease may be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break may be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of INDEL mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme may contain a mutation at D10, E762, H983, or D986, as well as a mutation at H840 or N863. In some instances, the dCas9 enzyme may contain a D10A or D10N mutation. Also, the dCas9 enzyme may contain a H840A, H840Y, or H840N. In some embodiments, the dCas9 enzyme may contain D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9(1.0)), and SpCas9 (K848A/K1003A/R1060A) (also referred to as eSpCas9(1.1)) variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

As described above, a gRNA may comprise a crRNA and a tracrRNAs. The gRNA can be configured to form a stable and active complex with a gRNA-mediated nuclease (e.g., Cas9 or dCas9). The gRNA contains a binding region that provides specific binding to the target genetic element. Exemplary gRNAs that may be used to target a region in a polynucleotide encoding a transcription factor selected from the group consisting of CIC, FOXO1, FOXP3, IKZF2, PRDM1, HIVEP2, SATB1, IRF4, T-bet, and GATA3 are listed in Table 2 below. A gRNA used to target a region in a polynucleotide encoding a transcription factor selected from the group consisting of CIC, FOXO1, FOXP3, IKZF2, PRDM1, HIVEP2, SATB1, IRF4, T-bet, and GATA3 may comprise a sequence selected from Table 2 below or a portion thereof. Table 2 also lists the editing efficiencies of each gRNA in two donors.

TABLE 2

| | | Editing | Editing |
|---|---|---|---|
| Transcription | | efficiency | efficiency |
| Factor | gRNA | donor1% | donor2% |
| CIC | TTGGGCCAGAGTACGATGCA (SEQ ID NO: 1) | 66.13 | 58.63 |
| CIC | CTCATGAACGGCCACCAGGT (SEQ ID NO: 2) | 76.15 | 68.34 |
| CIC | GGGTCCTGGAGCTCCATACT (SEQ ID NO: 3) | 71.41 | 53.26 |
| FOXO1 | TCATCCTGTTCGGTCATAAT (SEQ ID NO: 4) | 61.53 | 43.04 |
| FOXO1 | TAGCATTTGAGCTAGTTCGA (SEQ ID NO: 5) | 83.89 | 74.92 |
| FOXO1 | CATGAAGTCGGCGCTGACAG (SEQ ID NO: 6) | 65.77 | 38.81 |
| FOXP3 | GGTGCCTCCGGACAGCAAAC (SEQ ID NO: 7) | 70.84 | 58.02 |
| FOXP3 | AATGGTGTCTGCAAGTGGCC (SEQ ID NO: 8) | 76.18 | 74.9 |
| FOXP3 | ACCCAGGCATCATCCGACAA (SEQ ID NO: 9) | 60.59 | 76.27 |
| FOXP3 | TCATGGCTGGGCTCTCCAGG (SEQ ID NO: 10) | 65.97 | 54.86 |
| IKZF2 | ATGGGTCTTTCTATCCTATT (SEQ ID NO: 11) | 72.38 | 63.49 |
| IKZF2 | TCAGCAGTTTCCCTGCTAAT (SEQ ID NO: 12) | 83.61 | 80.51 |
| IKZF2 | GATAGAAAGACCCATTAGCA (SEQ ID NO: 13) | 81.9 | 67.24 |
| PRDM1 | GGGTAGTGAGCGTTGTACGA (SEQ ID NO: 14) | 33.79 | 28.37 |
| PRDM1 | TTCTGCTTCTTCAGCGGGTA (SEQ ID NO: 15) | 82.94 | 67.8 |
| PRDM1 | TCCAGAATGGAGTCGCAGGT (SEQ ID NO: 16) | 88.11 | 83.66 |
| HIVEP2 | GTTGATGAAACAGAGCACTT (SEQ ID NO: 17) | 78.52 | 77.45 |
| HIVEP2 | TGCCTCTCTCCGGATTGCCA (SEQ ID NO: 18) | 68.91 | 53.35 |
| HIVEP2 | GTTCCTTACTTCAGTCTCTA (SEQ ID NO: 19) | 55.58 | 55.28 |
| SATB1 | GCATCTGTCACGTAAGACAG (SEQ ID NO: 20) | 75.45 | 69.03 |
| SATB1 | CTGGATTCCACTTTCCAACC (SEQ ID NO: 21) | 76.32 | 65.37 |
| SATB1 | GTGTGCGACCATTGTTCGGG (SEQ ID NO: 22) | 82.94 | 78.65 |
| IRF4 | GACATTGGTACGGGATTTCC (SEQ ID NO: 23) | 76.46 | 63.54 |
| IRF4 | CTGATCGACCAGATCGACAG (SEQ ID NO: 24) | 56.64 | 42.48 |
| IRF4 | GTGTACAGGATTGTTCCTGA (SEQ ID NO: 25) | 76.97 | 67.01 |
| GATA3 | CTGGACGGCGGCAAAGCCCT (SEQ ID NO: 26) | 79.67 | 64.48 |
| GATA3 | GAGCTGTACTCGGGCACGTA (SEQ ID NO: 27) | 92.3 | 75.1 |
| GATA3 | GTACTGCGCCGCGTCCATGT (SEQ ID NO: 28) | 69.16 | 59.41 |

In some embodiments, the sequence of the gRNA or a portion thereof is designed to complement (e.g., perfectly complement) or substantially complement (e.g., 50%, 55%, 60%, 65%, 70%, 75%, 80%, 85%, 90%, 91%, 92%, 93%, 94% 95%, 96%, 97%, 98%, or 99% complement) the target region in the polynucleotide encoding the transcription factor. In some embodiments, the portion of the gRNA that complements and binds the targeting region in the polynucleotide is, or is about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more nucleotides in length. In some cases, the portion of the gRNA that complements and binds the targeting region in the polynucleotide is between about 19 and about 21 nucleotides in length. In some cases, the gRNA may incorporate wobble or degenerate bases to bind target regions. In some cases, the gRNA can be altered to increase stability. For example, non-natural nucleotides, can be incorporated to increase RNA resistance to degradation. In some cases, the gRNA can be altered or designed to avoid or reduce secondary structure formation. In some cases, the gRNA can be designed to optimize G-C content. In some cases, G-C content is between about 40% and about 60% (e.g., 40%, 45%, 50%, 55%, 60%). In some cases, the binding region can contain modified nucleotides such as, without limitation, methylated or phosphorylated nucleotides.

In some embodiments, CRISPR/Cas genome editing may be used to perform large (e.g., genome-wide) screens for transcription factors or genes regulated by transcription factors that are involved in the modulation of the stability of regulatory T cells. CRISPR/Cas system may use multiple gRNAs or gRNA libraries that target multiple different target regions in numerous polynucleotides with a high probability of altering the transcription of the targeted polynucleotides to a detectable degree. In some cases, the CRISPR/Cas system may provide at least two or more gRNAs, e.g., at least 2, 5, 10, 15, 20, 25, 30, 35, 40, 45, or 50 gRNAs. Thus, the CRISPR/Cas system may be used to target a large number of genes.

In some embodiments, the gRNA can be optimized for expression by substituting, deleting, or adding one or more nucleotides. In some cases, a nucleotide sequence that provides inefficient transcription from an encoding template nucleic acid can be deleted or substituted. For example, in some cases, the gRNA is transcribed from a nucleic acid operably linked to an RNA polymerase III promoter. In such cases, gRNA sequences that result in inefficient transcription by RNA polymerase III, such as those described in Nielsen et al., Science. 2013 Jun. 28; 340(6140):1577-80, can be deleted or substituted. For example, one or more consecutive uracils can be deleted or substituted from the gRNA sequence. In some cases, if the uracil is hydrogen bonded to a corresponding adenine, the gRNA sequence can be altered to exchange the adenine and uracil. This “A-U flip” can retain the overall structure and function of the gRNA molecule while improving expression by reducing the number of consecutive uracil nucleotides.

In some embodiments, the gRNA can be optimized for stability. Stability can be enhanced by optimizing the stability of the gRNA:nuclease interaction, optimizing assembly of the gRNA:nuclease complex, removing or altering RNA destabilizing sequence elements, or adding RNA stabilizing sequence elements. In some embodiments, the gRNA contains a 5′ stem-loop structure proximal to, or adjacent to, the region that interacts with the gRNA-mediated nuclease. Optimization of the 5′ stem-loop structure can provide enhanced stability or assembly of the gRNA:nuclease complex. In some cases, the 5′ stem-loop structure is optimized by increasing the length of the stem portion of the stem-loop structure.

gRNAs can be modified by methods known in the art. In some cases, the modifications can include, but are not limited to, the addition of one or more of the following sequence elements: a 5′ cap (e.g., a 7-methylguanylate cap); a 3′ polyadenylated tail; a riboswitch sequence; a stability control sequence; a hairpin; a subcellular localization sequence; a detection sequence or label; or a binding site for one or more proteins. Modifications can also include the introduction of non-natural nucleotides including, but not limited to, one or more of the following: fluorescent nucleotides and methylated nucleotides.

Also described herein are expression cassettes and vectors for producing gRNAs in a host cell. The expression cassettes can contain a promoter (e.g., a heterologous promoter) operably linked to a polynucleotide encoding a gRNA. The promoter can be inducible or constitutive. The promoter can be tissue specific. In some cases, the promoter is a U6, H1, or spleen focus-forming virus (SFFV) long terminal repeat promoter. In some cases, the promoter is a weak mammalian promoter as compared to the human elongation factor 1 promoter (EF1A). In some cases, the weak mammalian promoter is a ubiquitin C promoter or a phosphoglycerate kinase 1 promoter (PGK). In some cases, the weak mammalian promoter is a TetOn promoter in the absence of an inducer. In some cases, when a TetOn promoter is utilized, the host cell is also contacted with a tetracycline transactivator. In some embodiments, the strength of the selected gRNA promoter is selected to express an amount of gRNA that is proportional to the amount of Cas9 or dCas9. The expression cassette can be in a vector, such as a plasmid, a viral vector, a lentiviral vector, etc. In some cases, the expression cassette is in a host cell. The gRNA expression cassette can be episomal or integrated in the host cell.

Zinc Finger Nucleases (ZFNs)

“Zinc finger nucleases” or “ZFNs” are a fusion between the cleavage domain of FokI and a DNA recognition domain containing 3 or more zinc finger motifs. The heterodimerization at a particular position in the DNA of two individual ZFNs in precise orientation and spacing leads to a double-strand break in the DNA. In some embodiments of the methods described herein, ZFNs may be used to inhibit the expression of one or more transcription factors selected from the group consisting of CIC, FOXO1, FOXP3, IKZF2, PRDM1, HIVEP2, SATB1, IRF4, T-bet, and GATA3, i.e., by cleaving the polynucleotide encoding the transcription factor. In other embodiments, ZFNs may be used to knock out one or more genes regulated by one or more transcription factors selected from the group consisting of CIC, FOXO1, FOXP3, IKZF2, PRDM1, HIVEP2, SATB1, IRF4, T-bet, and GATA3.

In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain requires the 5′ edge of each binding site to be separated by about 5-7 bp. Exemplary ZFNs that may be used in methods described herein include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

ZFNs can generate a double-strand break in a target DNA, resulting in DNA break repair which allows for the introduction of gene modification. DNA break repair can occur via non-homologous end joining (NHEJ) or homology-directed repair (HDR). In HDR, a donor DNA repair template that contains homology arms flanking sites of the target DNA can be provided.

In some embodiments, a ZFN is a zinc finger nickase which can be an engineered ZFN that induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33.

TALENs

TALENS may also be used to inhibit the expression of one or more transcription factors selected from the group consisting of CIC, FOXO1, FOXP3, IKZF2, PRDM1, HIVEP2, SATB1, IRF4, T-bet, and GATA3. In other embodiments, TALENS may be used to knock out one or more genes regulated by one or more transcription factors selected from the group consisting of CIC, FOXO1, FOXP3, IKZF2, PRDM1, HIVEP2, SATB1, IRF4, T-bet, and GATA3. “TALENs” or “TAL-effector nucleases” are engineered transcription activator-like effector nucleases that contain a central domain of DNA-binding tandem repeats, a nuclear localization signal, and a C-terminal transcriptional activation domain. In some instances, a DNA-binding tandem repeat comprises 33-35 amino acids in length and contains two hypervariable amino acid residues at positions 12 and 13 that can recognize one or more specific DNA base pairs. TALENs can be produced by fusing a TAL effector DNA binding domain to a DNA cleavage domain. For instance, a TALE protein may be fused to a nuclease such as a wild-type or mutated FokI endonuclease or the catalytic domain of FokI. Several mutations to FokI have been made for its use in TALENs, which, for example, improve cleavage specificity or activity. Such TALENs can be engineered to bind any desired DNA sequence.

TALENs can be used to generate gene modifications by creating a double-strand break in a target DNA sequence, which in turn, undergoes NHEJ or HDR. In some cases, a single-stranded donor DNA repair template is provided to promote HDR.

Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55.

Meganucleases

“Meganucleases” are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric.

In some embodiments of the methods described herein, meganucleases may be used to inhibit the expression of one or more transcription factors selected from the group consisting of CIC, FOXO1, FOXP3, IKZF2, PRDM1, HIVEP2, SATB1, IRF4, T-bet, and GATA3, i.e., by cleaving in a target region within the polynucleotide encoding the transcription factor. In other embodiments, meganucleases may be used to knock out one or more genes regulated by one or more transcription factors selected from the group consisting of CIC, FOXO1, FOXP3, IKZF2, PRDM1, HIVEP2, SATB1, IRF4, T-bet, and GATA3.

In some instances, the meganuclease is naturally-occurring (found in nature) or wild-type, and in other instances, the meganuclease is non-natural, artificial, engineered, synthetic, or rationally designed. In certain embodiments, the meganucleases that may be used in methods described herein include, but are not limited to, an I-CreI meganuclease, I-CeuI meganuclease, I-MsoI meganuclease, I-SceI meganuclease, variants thereof, mutants thereof, and derivatives thereof.

Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

RNA-Based Technologies

Various RNA-based technologies may also be used in methods described herein to inhibit the expression of one or more transcription factors selected from the group consisting of CIC, FOXO1, FOXP3, IKZF2, PRDM1, HIVEP2, SATB1, IRF4, T-bet, and GATA3. In other embodiments, RNA-based technologies may be used to inhibit the expression of one or more genes regulated by one or more transcription factors selected from the group consisting of CIC, FOXO1, FOXP3, IKZF2, PRDM1, HIVEP2, SATB1, IRF4, T-bet, and GATA3. Examples of RNA-based technologies include, but are not limited to, small interfering RNA (siRNA), antisense RNA, microRNA (miRNA), and short hairpin RNA (shRNA).

RNA-based technologies may use an siRNA, an antisense RNA, a miRNA, or a shRNA to target a sequence, or a portion thereof, that encodes a transcription factor. In some embodiments, one or more genes regulated by a transcription factor may also be targeted by an siRNA, an antisense RNA, a miRNA, or a shRNA. An siRNA, an antisense RNA, a miRNA, or a shRNA may target a sequence comprising at least 10, at least 20, at least 30, at least 40, at least 50, at least 60, at least 70, at least 80, at least 90, or at least 100 contiguous nucleotides.

An siRNA may be produced from a short hairpin RNA (shRNA). A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. Expression of shRNA in cells is typically accomplished by delivery of plasmids or through viral or bacterial vectors. Suitable bacterial vectors include but not limited to adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA is exported from the nucleus, then processed by a protein called Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, leading to its destruction and an eventual reduction in the protein encoded by the mRNA. Thus, the shRNA leads to targeted gene silencing.

The shRNA or siRNA may be encoded in a vector. In some embodiments, the vector further comprises appropriate expression control elements known in the art, including, e.g., promoters (e.g., inducible promoters or tissue specific promoters), enhancers, and transcription terminators.

### IV. Methods of Treatment

Any of the methods described herein may be used to modify Treg cells obtained from a human subject. Any of the methods and compositions described herein may be used to modify Treg cells obtained from a human subject to treat or prevent a disease (e.g., cancer, an autoimmune disease, an infectious disease, transplantation rejection, graft vs. host disease or other inflammatory disorder in a subject).

Provided herein is a method of treating cancer in a human subject comprising: a) obtaining Treg cells from the subject; b) modifying the Treg cells using any of the methods provided herein to decrease the stability of the Treg cells; and c) administering the modified Treg cells to the subject, wherein the human subject has cancer. Also provided herein is a method of treating an autoimmune disease in a human subject comprising: a) obtaining Treg cells from the subject; b) modifying the Treg cells using any of the methods provided herein to increase the stability of the Treg cells; and c) administering the modified Treg cells to the subject, wherein the human subject has an autoimmune disease.

In some embodiments, Treg cells obtained from a cancer subject may be expanded ex vivo. The characteristics of the subject's cancer may determine a set of tailored cellular modifications (i.e., which transcription factors and/or genes regulated by the transcription factors to target), and these modifications may be applied to the Treg cells using any of the methods described herein. Modified Treg cells may then be reintroduced to the subject. This strategy capitalizes on and enhances the function of the subject's natural repertoire of cancer specific T cells, providing a diverse arsenal to eliminate mutagenic cancer cells quickly. Similar strategies may be applicable for the treatment of autoimmune diseases, in which the modified Treg cells would have improved stability.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The following examples are provided by way of illustration only and not by way of limitation. Those of skill in the art will readily recognize a variety of non-critical parameters that could be changed or modified to yield essentially the same or similar results.

### Example 1—Arrayed Screens and Single-Cell RNA Sequencing

Human Blood

Whole blood of healthy donors was collected from human donors in blood bags (anticoagulant citrate phosphate dextrose salutation USP; Fenwal) with approval by the UCSF Committee on Human Research.

Treg Isolation and Culture

Whole human blood was processed within 24 hours after donation. Peripheral blood mononuclear cells (PBMCs) were isolated by Ficoll-Paque PLUS (GE Healthcare) or Lymphoprep (Stemcell Technologies) gradient centrifugation in SepMate50 tubes. After centrifugation (1,200 g, 10 minutes) the PBMC layer was removed and the cells were washed with EasySep buffer (PBS containing 2% FBS and 1 mM EDTA). CD4+ T cells were enriched with the EasySep Human CD4+ T-cell enrichment kit (Stemcell Technologies).

Pre-enriched CD4+ T cells were stained with the following antibodies: αCD4-PerCp (SK3; TONBO Biosciences), αCD25-APC (BC96; TONBO Biosciences), αCD127-PE (R34-34; TONBO Biosciences), αCD45RA-violetFluor450 (HI100; TONBO Bio-sciences), and αCD45RO-FITC (UCHL1; TONBO Biosciences). CD4+CD25hiCD127low Tregs or CD4+CD25-CD127high effector T cells were isolated using a FACS Aria Illu (Becton Dickinson). Treg purity was regularly >97%.

Isolated Tregs were suspended in complete Roswell Park Memorial Institute (cRPMI), consisting of RPMI-1640 (Sigma) supplemented with 5 mM 4-(2-hydroxyethyl)-1-piperazineethanesulfonic acid (HEPES, Gibco), 2 mM Glutamine (Gibco), 50 μg/mL penicillin/streptomycin (Gibco), 5 mM nonessential amino acids (Gibco), 5 mM sodium pyruvate (Gibco), and 10% fetal bovine serum (FBS, Atlanta Biologicals). These cells were immediately stimulated on anti-CD3 coated plates [coated overnight with 10 μg/mL αCD3 (UCHT1, Tonbo Biosciences)] in the presence of 5 μg/mL soluble anti-CD28 (CD28.2, Tonbo Biosciences).

For arrayed and pooled Cas9 RNP screens, Tregs were expanded ex vivo for 11 days before nucleofection. Freshly isolated Tregs were cultured in complete RPMI with anti-CD3/CD28-coated beads in a 1:1 ratio. Starting day 2 of culture, 300 IU/mL IL-2 were added and replenished every 48 hrs. On day 9 of Treg expansion, cells were re-stimulated in 48-well plates coated overnight with 10 μg/ml anti-CD3 (UCHT1; TONBO Biosciences) and 5 μg/mL anti-CD28 (CD28.2; TONBO Biosciences) for 48 hours.

For single-cell RNA sequencing (scRNA-seq) experiments, freshly isolated Tregs were stimulated for 48 hrs in cRPMI with 5 μg/mL anti-CD28 (CD28.2; TONBO Biosciences) on plates coated with 10 μg/mL anti-CD3 (UCHT1; TONBO Biosciences) prior nucleofection.

Generation of Homology-Directed Repair (HDR)-Templates

Plasmids encoding for GFP and 500 bp long homology arms to either tag RAB11 with GFP or replace exon1 of FOXP3 with GFP were generated by introducing gene blocks (IDT) into the vector pUC19 via Gibson assembly. The inserts were PCR-amplified using KAPA HiFi HotStart ReadyMix (2×). The PCR product was purified with SPRI beads and resuspended in water.

Cas9 RNP Assembly and Nucleofection

80 μM crRNA (Dharmacon) and 80 μM tracrRNA (Dharmacon) were mixed in a 1:1 ratio and incubated for 30 minutes at 37° C. to generate 40 μM crRNA:tracrRNA duplexes. An equal volume of 40 μM S. pyogenes Cas9-NLS (Macrolabs, Berkeley) was slowly added to the crRNA:tracrRNA and incubated for 15 minutes at 37° C. to generate 20 μM Cas9 RNPs. For each reaction, roughly 150,000-300,000 stimulated T cells were pelleted and re-suspended in 20 μL P3 buffer. 3 μl 20 μM Cas9 RNP mix was added directly to these cells and the entire volume transferred to the 96-well reaction cuvette. For knock-in experiments 100 ng of HDR-template was added. HDR-template was heated for one minute in boiling water to generate single-stranded DNA. Tregs were electroporated using program EH-115 on the Amaxa 4D-Nucleofector (Lonza). 80 μL pre-warmed, complete RPMI was added to each well after nucleofection, and the cells were allowed to recover for 30 minutes at 37° C. before restimulation.

Arrayed Cas9 RNP Screen

Electroporation of cells was performed using the Amaxa P3 Primary Cell 96-well Nucleofector kit and 4D-Nucleofecter (Lonza). Each reaction contained 200,000 expanded Tregs, 3 μL of the respective RNP and 20-100 ng of a non-targeting ssDNA (Ultramer; IDT) to enhance editing efficiency.

Prior to FACS staining, cells were stimulated with PMA/Iono/Brefeldin (cell activation cocktail with Brefeldin; Biolegend) for 6 hrs at 37° C. Cells were stained extracellularly with anti-CD25-PeCy7 (clone: M-A251; Biolegend) and ghost dye 510 (Tonbo Biosciences) on ice for 30 min. After performing Fix&Perm (Foxp3 staining buffer set; Biolegend) for 30 min at RT cells were stained intracellularly for the following protein markers: Foxp3-AF488 (clone: 206D; Biolegend), IFNg-BD Horizon 450 (clone: B27; Biolegend), IL-10-PE (clone: JES3-9D7; BD Pharmingen), IL-2-BV650 (clone: MQ1-17H12; Biolegend), Helios-Percy-Cy5.5 (clone: 22F6; Biolegend), CTLA-4-APC (clone: L3D10; Biolegend), IL-17a-AF700 (clone: BL168; Biolegend), and IL-4-APC-Cy7 (clone: MP4-25D2; Biolegend). Intracellular stainings were performed in 80% Perm buffer (Foxp3 staining buffer set; Biolegend) and 20% BDHorizon Brilliant Stain Buffer (BD Biosciences) for 30 min at RT. Cells were acquired on a BD Fortessa X20 Dual instrument (Becton Dickison). FACS data were analyzed using FlowJo to visualize multidimensionally scaffold

Single-Cell RNA Sequencing (scRNA-Seq)

CRISPR-edited Tregs were stimulated with PMA/Iono (cell activation cocktail without Brefeldin; Biolegends) for 6 hrs in RPMI complete with 300 IU/ml IL-2. Cells were washed twice with PBS and resuspended to a final concentration of 1000-2000 cells/μL in PBS containing 0.4% BSA. For pooled scRNA-seq, cells of two donors were mixed in a 1:1 ratio and 30,000 cells loaded onto a chip (10× Genomics). For experiments with cells of one single donor, 10,000-12,000 cells were applied. The RNA capture, barcoding, cDNA and library preparation were performed according to the manufacturer's recommendations. Libraries were sequenced either on HiSeq4000 or NovaSeq S4 instruments (Illumina). All scRNA-seq experiments were performed with cells of male donors.

### Example 2—Pool Cas 9 RNP Screens

Treg Isolation and Culturing

For the isolation of human Tregs, PBMCs were isolated from leukoreduction filters from whole blood collections (Blood Centers of the Pacific, San Francisco) using density gradient centrifugation. Human CD4+ T cells were purified by negative magnetic selection (EasySepTM™) followed by sorting of CD4+, CD25+, CD127low cells using a FACSAria II sorter (BD).

Freshly isolated Tregs were activated with anti-CD3/anti-CD28-coated microbeads (Dynabeads CTS) at a 1:1 bead-to-cell ratio and kept under conventional cell culture conditions at 37° C. with 5% CO₂. On days 2, 5, and 7, IL-2 was added. On day 9, Tregs were re-stimulated with plate bound anti-CD3 and soluble anti-CD28 (5 μg/mL; TONBO biosciences) in the presence of IL-2 for 48 h.

Pooled RNP Screen

For the generation of the gRNA, crRNA and tracrRNA were mixed in a 1:1 ratio. After incubation for 30 min at 37° C., an equal volume of 40 μM Cas9 solution was added and incubated at 37° C. for 15 min. For the pooled RNP-Mix, all RNPs were prepared separately and mixed in the end.

15 10⁶ cells were suspended in 100 μL Lonza P3 electroporation buffer. The cell suspension was mixed with 20 μL RNP-Mix and 6.6 μL electroporation enhancer (fragmented DNA). The solution was transferred to an electroporation cuvette and cells were electroporated using the program EH-115 on the Amaxa 4D-Nucleofector.

400 μL of pre-warmed, complete RPMI was added immediately after electroporation and the cells were allowed to recover for 30 min at 37° C. Cells were then transferred to a 48-well plate with 10⁶ cells per well, 300 U/mL IL-2 and re-stimulated with anti-CD3/anti-CD28-coated microbeads (1:1 bead-to-cell ratio). After 24 hrs, Tregs were adjusted according to the Treg expansion scheme and cultured for 72 h in media with either IL-12 (10 ng/mL; Fischer Scientific) plus IL-2, IL-4 (10 ng/mL; TONBO biosciences) plus IL-2, IL-6 (10 ng/mL; Fischer Scientific) plus IL-2, IFN-γ (10 ng/mL; TONBO biosciences) plus IL-2 or IL-2 alone. Where indicated, 300 U/mL of recombinant human IL-2 was added to the media.

Antibodies and Flow Cytometry

The following fluorescent dye-conjugated antibodies against surface and intracellular antigens were used: live/dead-APC-Cy7, anti-FOXP3-AF488, anti-CD25.APC, anti-CTLA4-PE, anti-IL-2-BV650, and anti-IFNγ-V450.

To determine cytokine expression cells were stimulated in media containing phorbol 12-myristate 13-acetate, ionomycin and brefeldin-A (Cell Activation Cocktail with Brefeldin A; Biolegend) for 4.5 hrs before staining. After stimulation, cells were placed on ice and stained with an amine-reactive exclusion-based viability dye (Ghost dye 780; TONBO biosciences) and with antibodies against cell-surface antigens for 30 min. Intracellular staining was carried out using the FOXP3 staining kit (Biolegend). Fixed and permeabilized cells were stained with the specific anti-FOXP3 and anti-cytokine antibodies at RT for 30 min. All samples were sorted with a FACSAria II sorter (BD) based on the expression of FOXP3, CTLA4, and IFN-γ. Between 50×10³ and 200×10³ cells of the negative and positive subpopulations for each marker were collected.

Isolation and Amplification of Genomic DNA

Cells were incubated overnight in 400 μL lysis buffer (0.5% SDS, 50 mM Tris, pH 8, 10 mM EDTA) at 66° C. For RNA digestion, samples were treated with 0.2 mg/mL RNAse at 37° C. for 1 hr. Incubation at 45° C. with 0.5 mg/mL Proteinase K for 1 hr was used for protein digestion. For the extraction of the DNA, one volume of Phenol:Chloroform:Isoamyl Alcohol (25:24:1) was added to the sample. After a short centrifugation, the aqueous phase was transferred and 600 μL isopropanol and 40 μL of 3 M sodium acetate were added. DNA precipitated after 30 min at −80° C. and 30 min of centrifugation (max speed) at 4° C. The DNA containing pellet was resuspended in TE buffer.

For the amplification of targeted regions, the CleanPlex™ Targeted Library Kit from Paragon Genomics was used. The kit allows amplification of all target regions in a multiplex PCR reaction and generates a library for Next-Generation Sequencing in a second round of PCR. Purification between these steps with a Digestion Reagent and magnetic beads yields a high purity library free of nonspecific PCR products. All steps were performed according to manufacturer's instructions, while using 40 ng of extracted genomic DNA for each initial PCR reaction.

The size of all amplicons ranged between 150 and 200 bp and the position of gRNA cut site was at least 30 bp away from the 3′ and 5′-end for most of the samples. Some exceptions are ZNF335 (20 bp), NR4A1 (15 bp), FOXO3 (21 bp), and ZNF831 (21 bp). For quality control, for each target, an amplicon, which was 500 to 1000 bp away from the respective cut site and should remain unmodified, was included.

Next-Generation Sequencing (NGS)

Prior to sequencing, purity was tested on an Agilent Bioanalyzer. Sequencing was performed on the MiniSeq (Illumina) platform in combination with the MiniSeq System High-Output Kit (150 cycles).

Data Analysis

All flow cytometry data was analyzed using FlowJo software (TreeStar). The web tool CRRISPResso was used for the analysis of genome editing outcomes from deep sequencing data. This tool gives precise information about the number and position of deletions, mutations, and insertions in deep sequencing data. For detailed analysis of the CRRISPResso output, R version 3.2.5 with the packages “ggplot”, “limma” and “vegan” was used. The batch effect removal algorithm from the “limma” package was used. All reads with in frame deletions and insertions shorter than 9 bp were excluded. For easier comparison of the data points, for each amplicon, the loge fold change of the editing efficiency between the sorted positive and negative subpopulations was calculated.

### Example 3—Pooled Cas9 Ribonucleoprotein (RNP) Complex Screen Targeting Treg Cell-Specific Transcription Factors

Current understanding of transcriptional regulation of human Treg cell stability and maintenance is still very scarce partially because it was very challenging to genetically manipulate these cells. A method of pooled Cas9 RNP editing targeting many different transcription factors in one assay to assess their influence on the expression levels of canonical Treg cell and effector T (Teff) markers may be used. Transcription factors may be chosen based on differential comparison of RNA sequencing data sets identifying transcription factors that are preferentially expressed in Treg cells (e.g., FOXP3 and IKZF2) compared to other CD4+ T cell subsets.

Each transcription factor may be targeted with one RNP complex in a pooled approach. The individual RNP complex may be assembled separately. The pool of RNP complexes targeting the many transcription factors may be directly nucleofected in ex vivo expanded human Treg cells. The pool of edited cells may be challenged with different pro-inflammatory cytokines (e.g., IL-4, IL-6, IL-12, IFNγ) to see the full spectrum of destabilization or stabilization before cells were sorted based on their expression levels of the Treg cell markers, e.g., Foxp3, CTLA-4, or the effector cytokine IFNγ. Stability of the Treg cells may be assessed using data from the arrayed screen and the FACS readout. Some of the FACS markers used are canonical Treg cell signature proteins. Some of the FACS markers are proteins that are normally not expressed in Treg cells, but are expressed under pro-inflammatory challenges (i.e., using pro-inflammatory cytokines (e.g., IL-4, IL-6, IL-12, IFNγ)). Using the FACS markers, the loss of Treg cell canonical markers and/or gain of pro-inflammatory markers were assessed and analyzed to determine the change in Treg cell stability.

DNA of the sorted cells may be recovered and enrichment of specific indels in the Treg cells may be determined by multiplexed amplicon PCR followed by deep-sequencing. In some embodiments, pooled RNP complex screens may be performed in primary human Treg cells. In some embodiments, novel phenotypes may be detected, i.e., transcription factor IKZF2 depletion protecting Treg cells from producing IFNγ.

### Example 4—Arrayed Cas9 RNP Complex Screen Targeting Treg Cell-Specific Transcription Factors for Detailed Analysis

To analyze the Treg cells with one or more transcription factors knocked out in more detail, an arrayed Cas9 RNP complex screen was performed targeting various transcription factors. Each transcription factor was targeted with different guide RNAs in an arrayed format in two donors. Cell stability was determined by a multi-color FACS panel based on Treg cell markers like Foxp3, Helios, CTLA-4, CD25, IL-10, and effectors such as cytokines typically associated with effector T cell subsets like IL-2, IFNγ, IL-17a, and IL-4 (see Table 1). To see the full spectrum of destabilization, it was hypothesized that the cells need to undergo a pro-inflammatory challenge. To assay this phenotype, the cells were treated for 3 days with high doses of IL-12.

It was hypothesized that all nine FACS markers need to be integrated to see the full spectrum of destabilized subpopulations within one knockout condition. To visualize Treg cell subpopulations, a Cytof analysis pipeline was adopted for large scale FACS data. By applying the Cytof pipeline scaffold to FACS data, diverse subpopulations of Treg cells and different destabilization phenotypes were distinguished. The patterns were compared in different transcription knock-out conditions. RNP complexes targeting the transcription factors Foxp3, IRF4, PRDM1, and FOXO1 resulted in strong deregulation of several FACS markers. Further changes in Treg cell stability could be detected in GATA3, HIVEP2, SATB1, and CIC targeted cells. As further controls, IKZF2 and TBX21 knocked-out Treg cells were included in the downstream analysis (single cell RNA-sequencing). The individual editing efficiencies of various guide RNAs in the screen achieved from 40% to 80%.

### Example 5—Single-Cell RNA-Sequencing Analysis for Treg Cell Destabilization

Single-cell RNA-sequencing (scRNA-seq) was used to analyze the results in more detail. The workflow was as described for the arrayed screen and also included unstimulated and IL-12 treated conditions for each transcription knock-out condition. Different subpopulations (“clusters”) in the Treg cell scRNA-seq data were distinguished. Based on the gene lists defining the individual clusters, IL-12 treated transcription factor knocked-out Treg cells showed a higher diversity and a higher destabilization. Transcription factor-specific effects were observed in the destabilization phenotypes. For example, IL-12-treated conditions clustered: c6 (enriched for FOXP3, IRF4, FOXO1, PRDM1, and SATAB1 knock-outs) showed a very distinct pattern of high cytokine secretion; and c7 (enriched for IRF4 knock-out) expressed low levels of IL2RA and switched from oxidative phosphorylation to glycolysis.

### Example 6—Deregulation of Several Markers in FOXP3 Knocked-Out Treg Cells

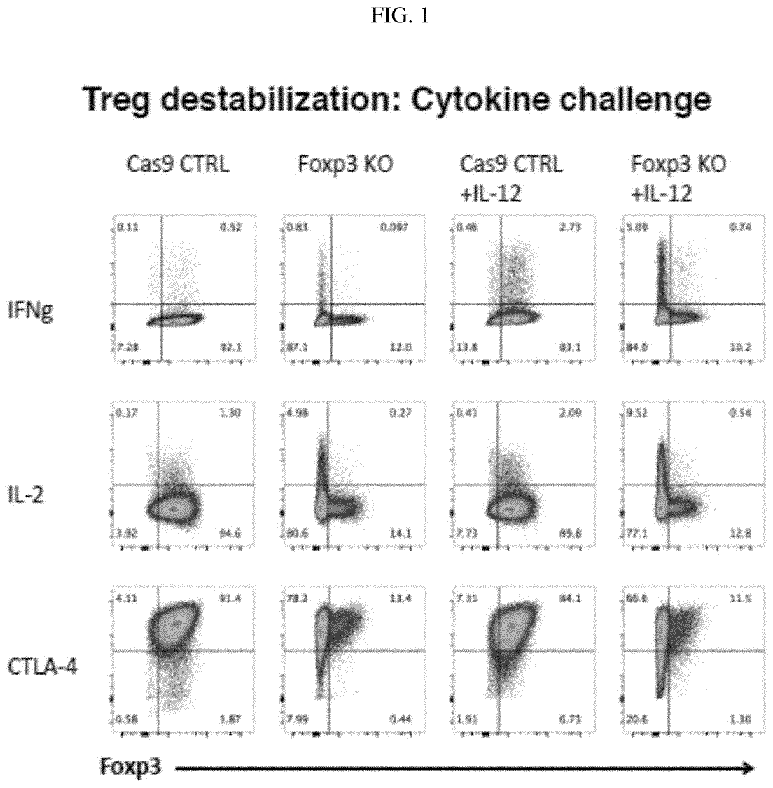

FOXP3 knocked-out Treg cells were acquiring a destabilized phenotype as characterized by the loss of marker CTLA-4 and gain of effector cytokine secretion, e.g., IL-2 and IFNγ. These phenotypes can be accelerated by IL-12 proinflammatory challenge. FIG. 1 shows deregulation of several Treg/Teff markers in FOXP3 knocked-out Treg cells and that the phenotype can be exacerbated by pro-inflammatory IL-12 challenge.

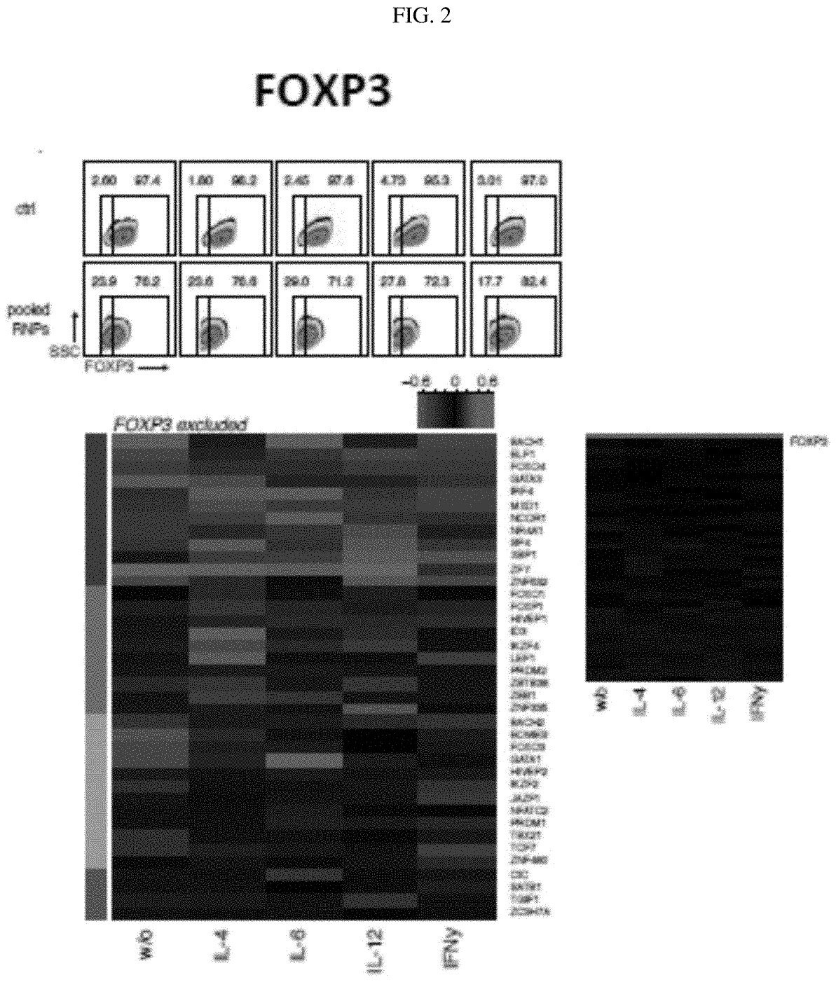

FIG. 2 further shows a heatmap summarizing the results of FOXP3 screen (FOXP3 positive control excluded) for 39 transcription factors in all the tested stimulating conditions. Results were clustered based on kₘₑₐₙₛ=4. Top section of the bar on the left: Editing these transcription factors showed the strongest reduction in FOXP3 expression levels (strongest enrichment of edits in exTregs compared to Tregs). Second section of the bar on the left: Editing in these transcription factors showed a slight reduction of FOXP3 expression. Third section: Editing these transcription factors showed no clear phenotype. Last section: Editing these transcription factors showed protection from Foxp3 loss, stabilizing FOXP3 expression. Top of FIG. 2 shows representative FACS results of FOXP3 screen in one donor. Control and pooled knocked-out Treg cells were challenged with IL-4, IL-6, IL-12, or IFNγ. Gating strategy to isolate: Treg cells (FOXP3+) and ex-Treg cells (FOXP3−).

### Example 7—CTLA-4 and IFNγ Expressions

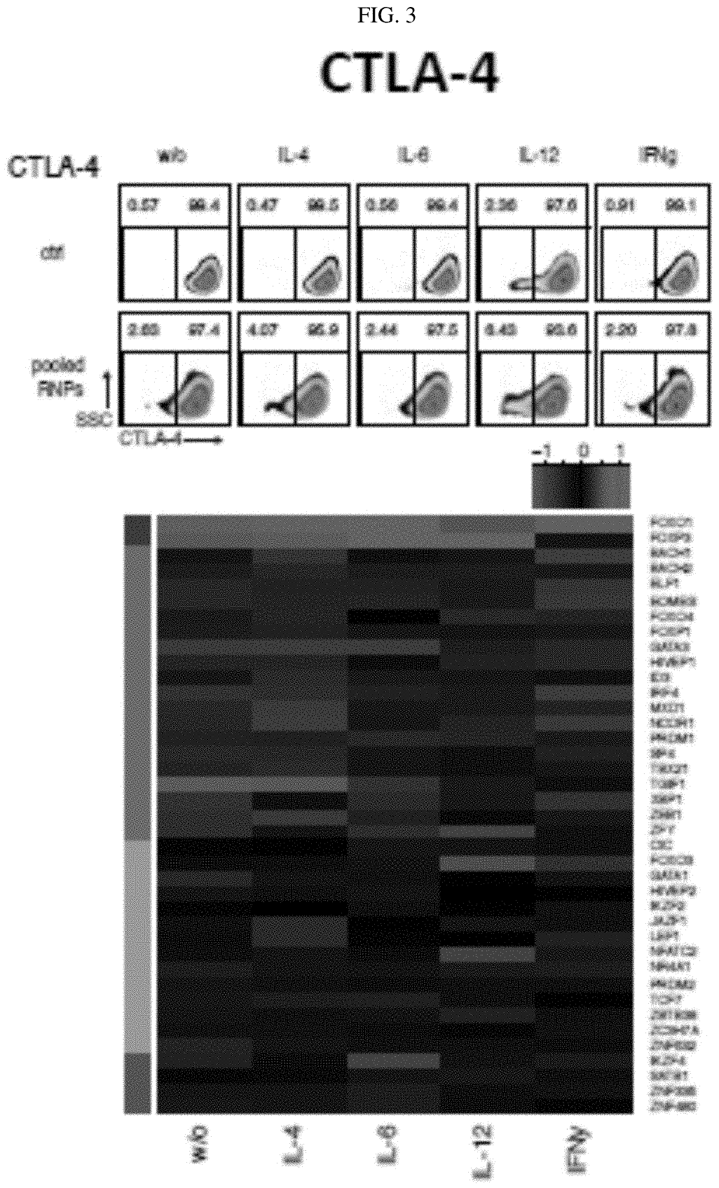

FIG. 3 shows a heatmap summarizing the results of CTLA4 screen for 39 transcription factors in all the tested stimulating conditions. The results were clustered based on kₘₑₐₙₛ=4. Top section of the bar on the left: Editing these transcription factors showed the strongest reduction in CTLA4 expression levels (strongest enrichment of edits in exTregs compared to Tregs). Second section of the bar on the left: Editing in these transcription factors showed a slight reduction of CTLA4 expression. Third section of the bar on the left: Editing these transcription factors showed no clear phenotype. Last section of the bar on the left: Editing these transcription factors showed protection from CTLA4 loss, stabilizing CTLA4 expression. Top of FIG. 3 shows representative FACS results of CTLA-4 screen in one donor. Control and pooled knocked-out Treg cells were challenged with IL-4, IL-6, IL-12, or IFNγ. Gating strategy to isolate: Treg cells (CTLA4+) and ex-Tregs (CTLA4−).

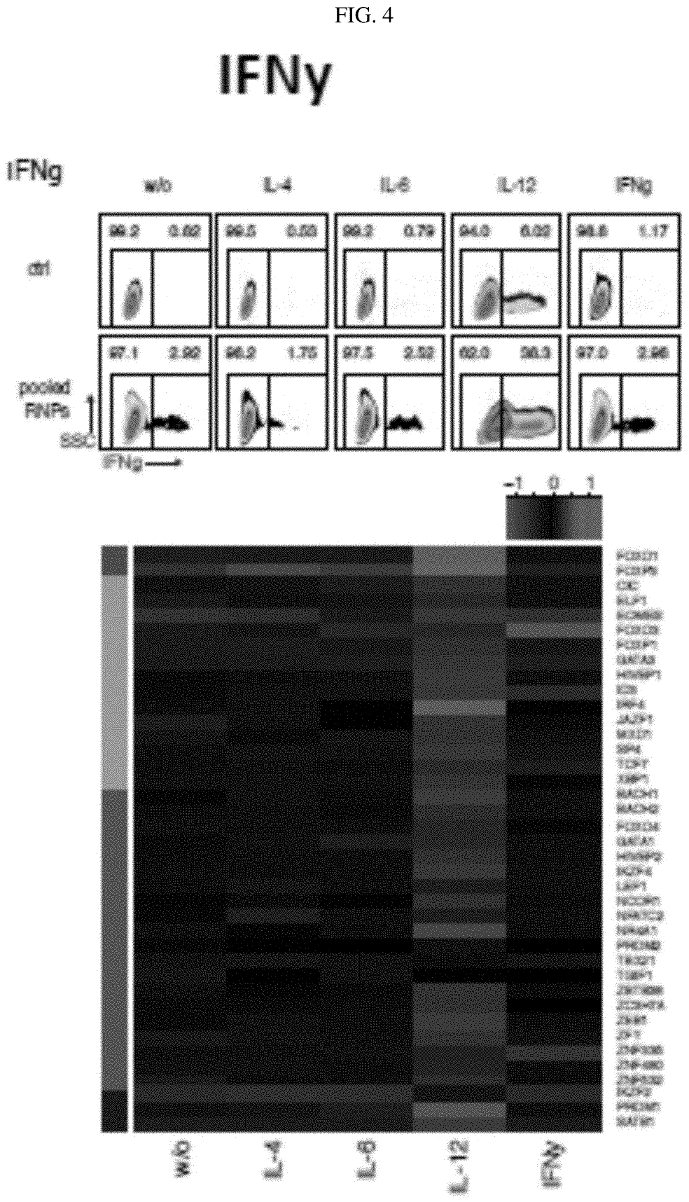

FIG. 4 shows a heatmap summarizing the results of IFNγ screen for 39 transcription factors in all the tested stimulating conditions. Results were clustered based on kₘₑₐₙₛ=4. Top section of the bar on the left: Editing these transcription factors showed the strongest induction of IFNγ. Second section of the bar on the left: Editing these transcription factors showed no clear phenotype. Third section of the bar on the left: Editing these transcription factors showed protection from FOXP3 loss, stabilizing FOXP3 expression. Last section of the bar on the left: strongest protection of IFNγ induction in Treg cells by editing these transcription factors. Overall, IL-12 stimulation was able to induce IFNγ expression (known positive feedback loop), but not in TBX21 knockout (negative control). Top of FIG. 4 shows representative FACS results of IFNγ screen in one donor. Control and pooled knocked-out Treg cells were challenged with IL-4, IL-6, IL-12, or IFNγ. Gating strategy to isolate: Tregs (IFNg−) and ex-Tregs. (IFNg+).

### Example 8—Comparison of Pooled and Arrayed Screens

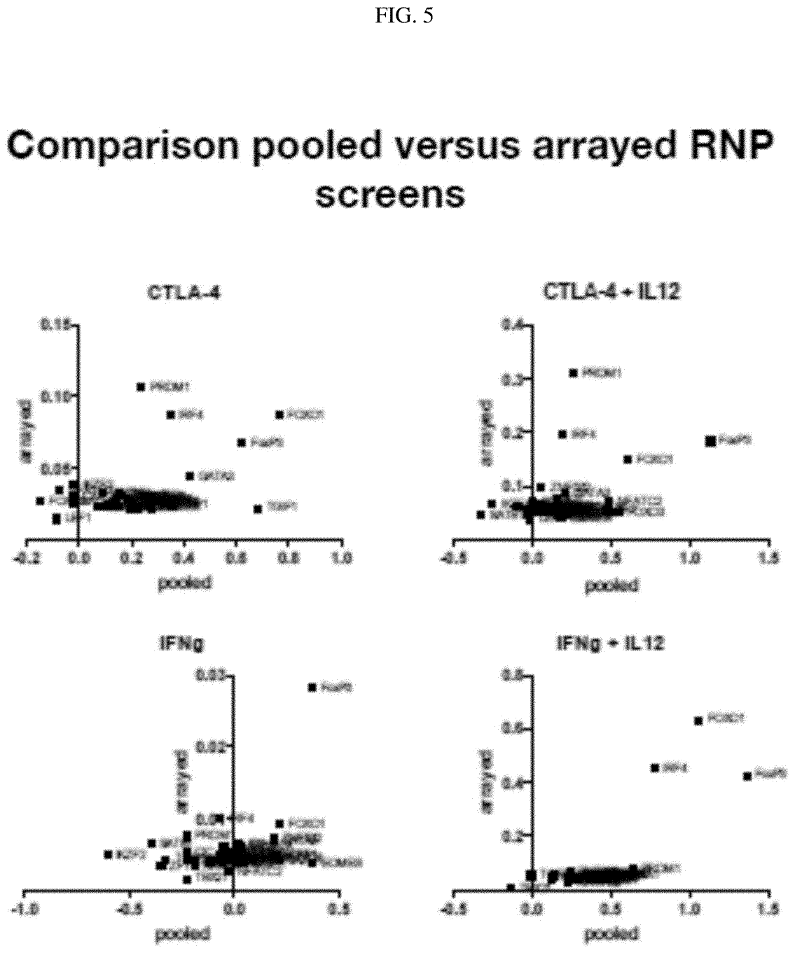

Correlation of the phenotypes in the pooled (log 2 fold change editing efficiencies in exTreg/Treg fraction) versus the arrayed (% protein expression changes based on FACS) screen based on CTLA-4 and IFNγ expression with and without IL-12 challenge was tested (FIG. 5). A real linear correlation was not detected, but the strongest hits, e.g., PrDM1, FOXP3, IRF4, and FOXO1 in the CTLA4+IL12 conditions can be clearly detected in the pooled and arrayed screens.

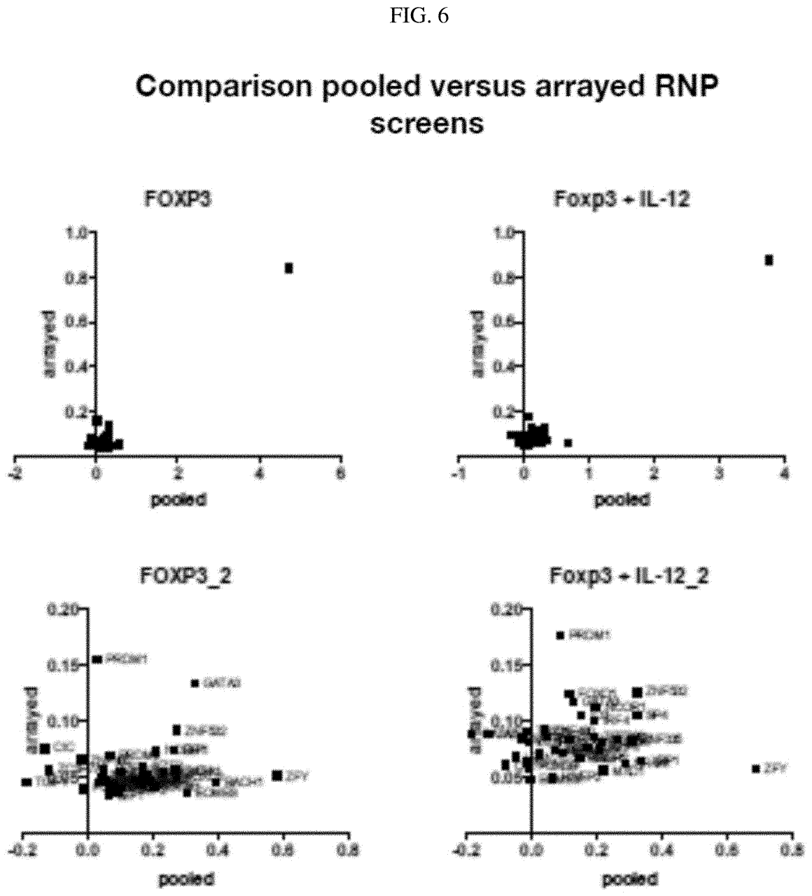

Further, FIG. 6 shows a comparison of the results of pooled (log 2 fold change editing efficiencies in exTreg/Treg fraction) versus arrayed (% protein expression changes based on FACS) screens based on FOXP3 expression with and without IL-12 challenge. Bottom panels: Results of editing in the FOXP3 locus (positive control) excluded.

### Example 9—Multidimensional FACS Analysis

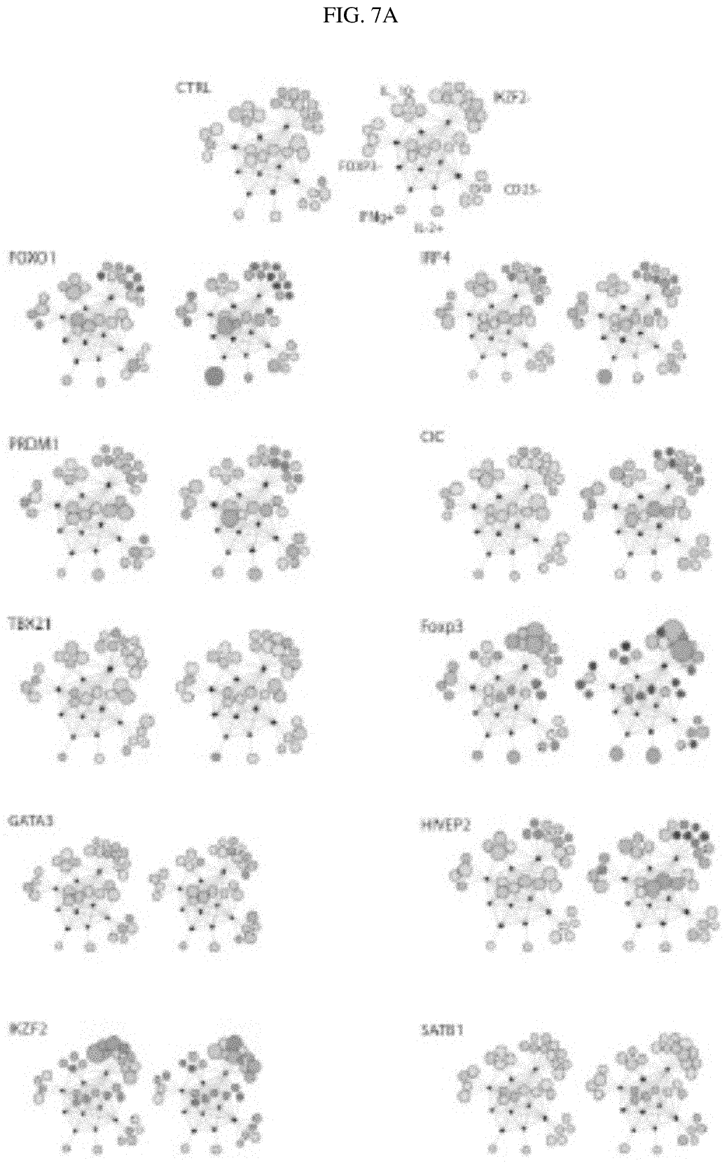

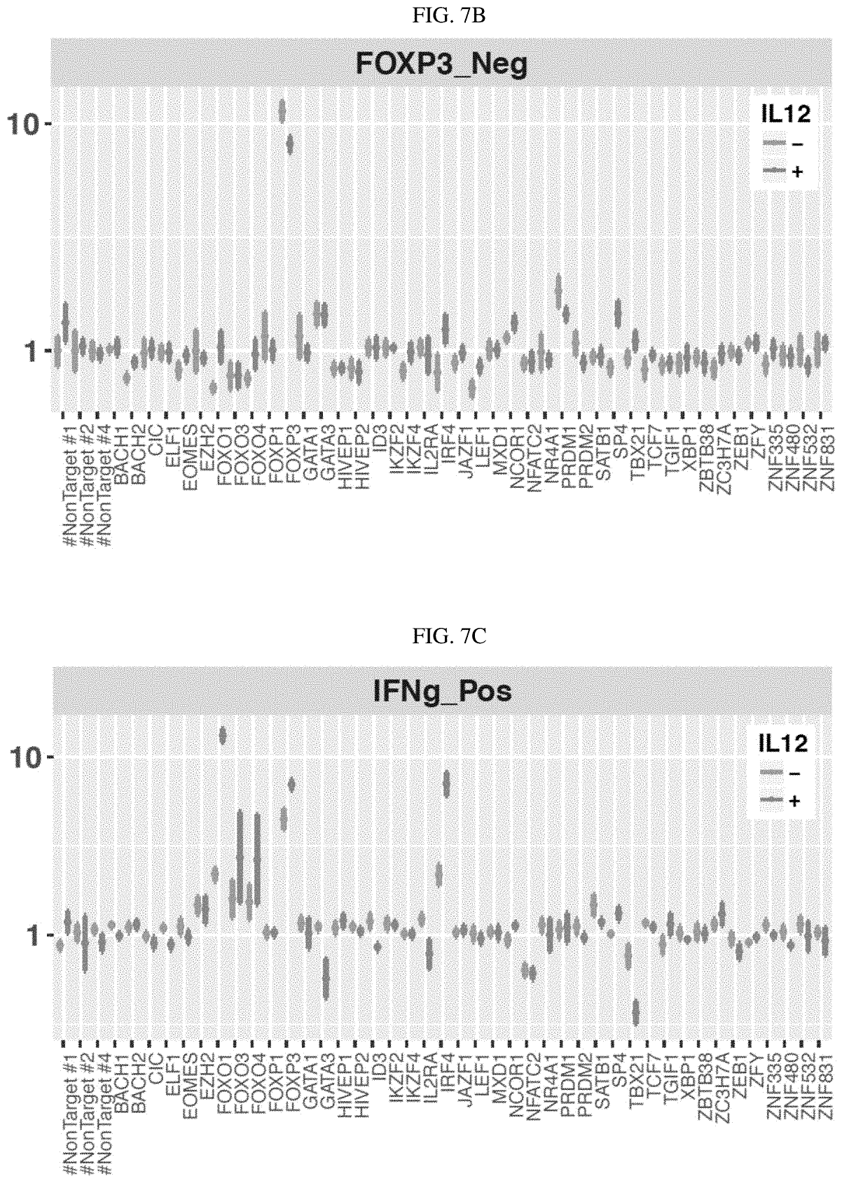

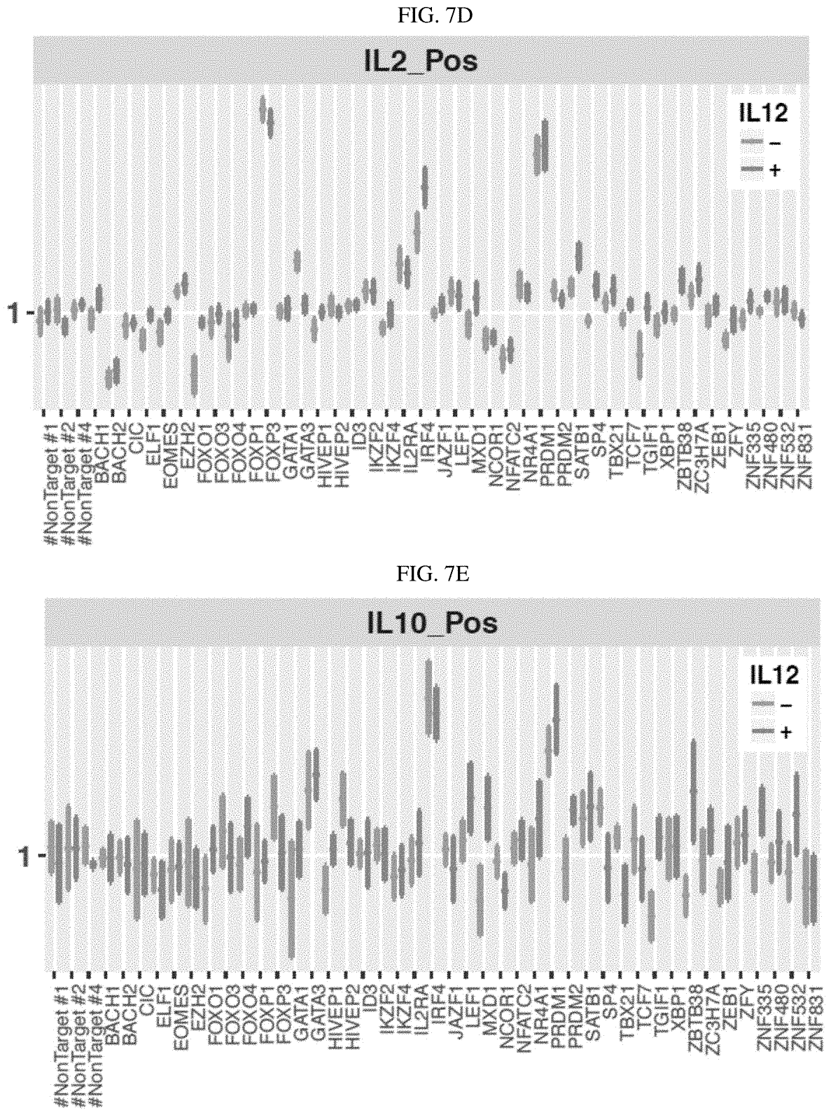

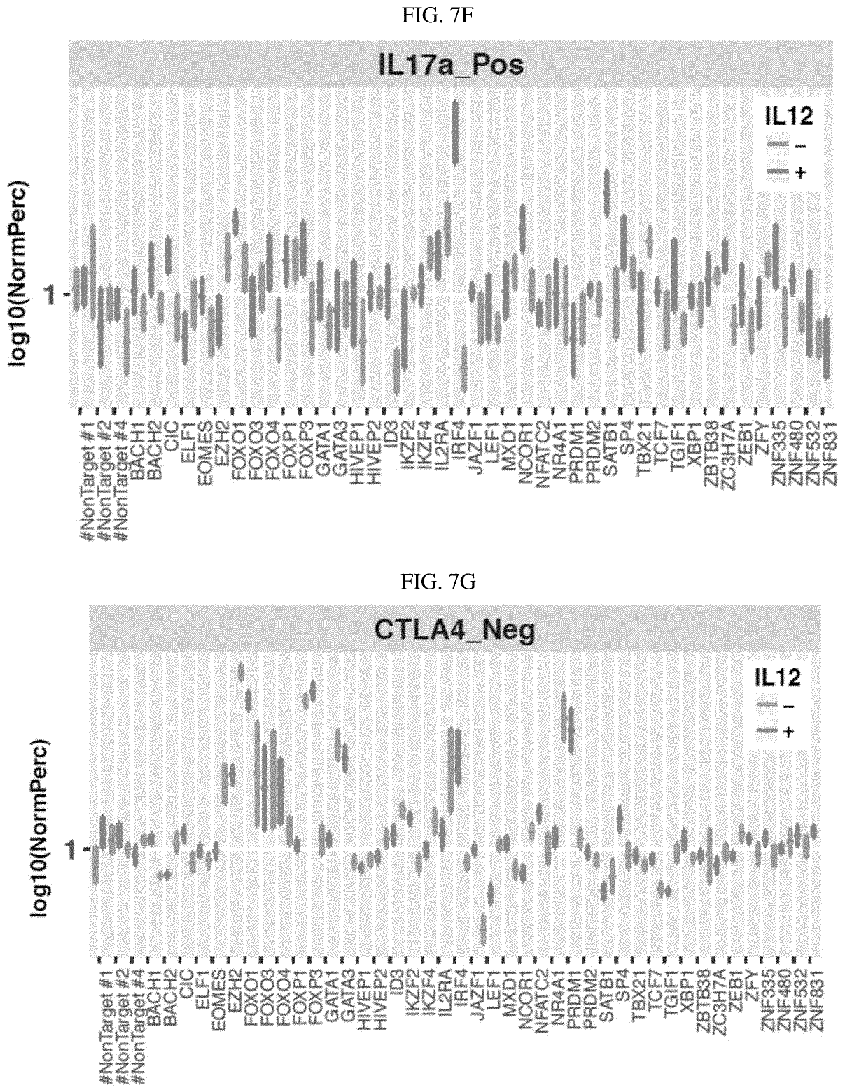

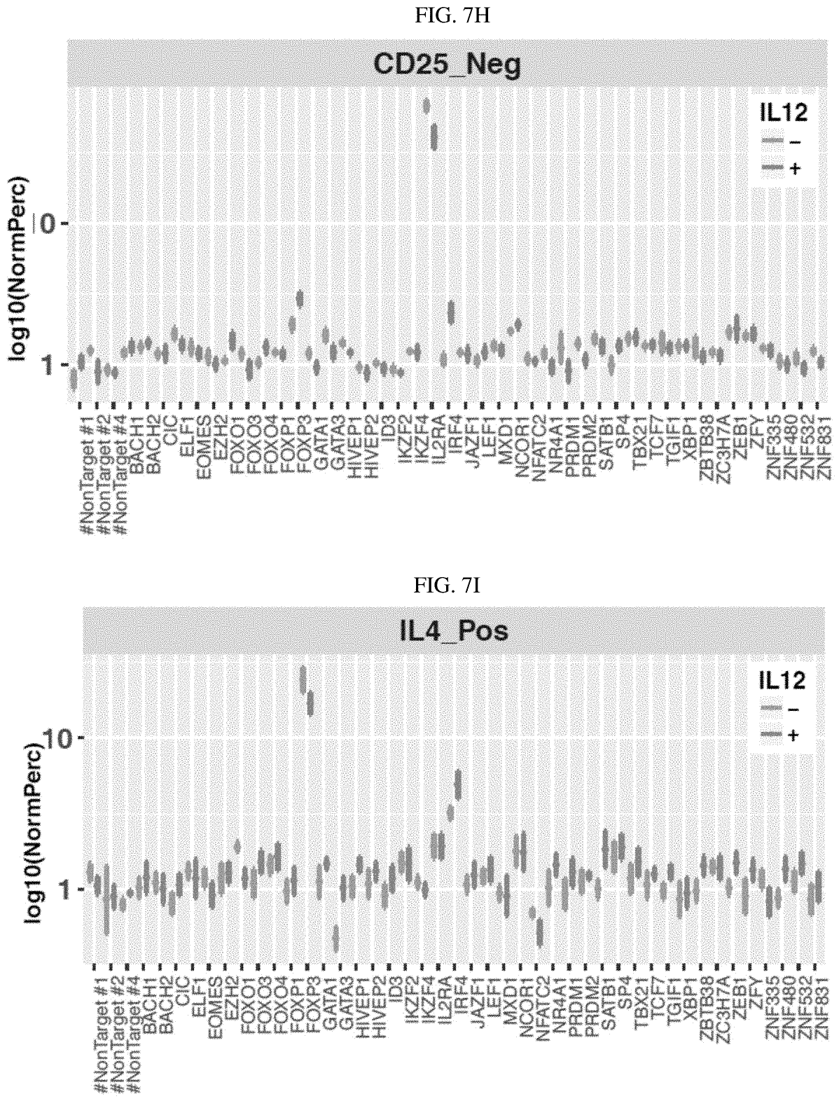

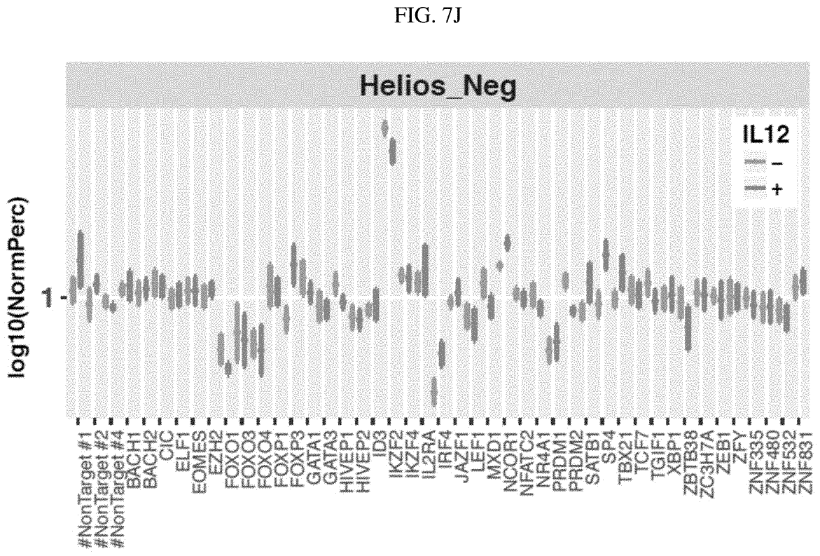

A Cytof-analysis pipeline for the multi-dimensional analysis of FACS data was used (FIG. 7A). It allows different subpopulations of cells in the respective knock-outs to be distinguished. All transcription factors (except TBX21—negative control) changes in sub-populations compared to the control, which are probably different subsets of destabilized Treg cells. Left of FIG. 7A: transcription factor knock-out without IL12. Right: Same transcription factor knock-out with IL12 stimulation. Size of bubbles indicates number of cells in the respective bubble. Blue: reduction of the markers that define this bubble. Red: upregulation of the respective markers. Black: landmark nods (reference points) to cluster all the different conditions in the same way. FIGS. 7B-7J further provide FACS data analysis.

### Example 10—Clustering of Single Cell RNA-Sequencing Data

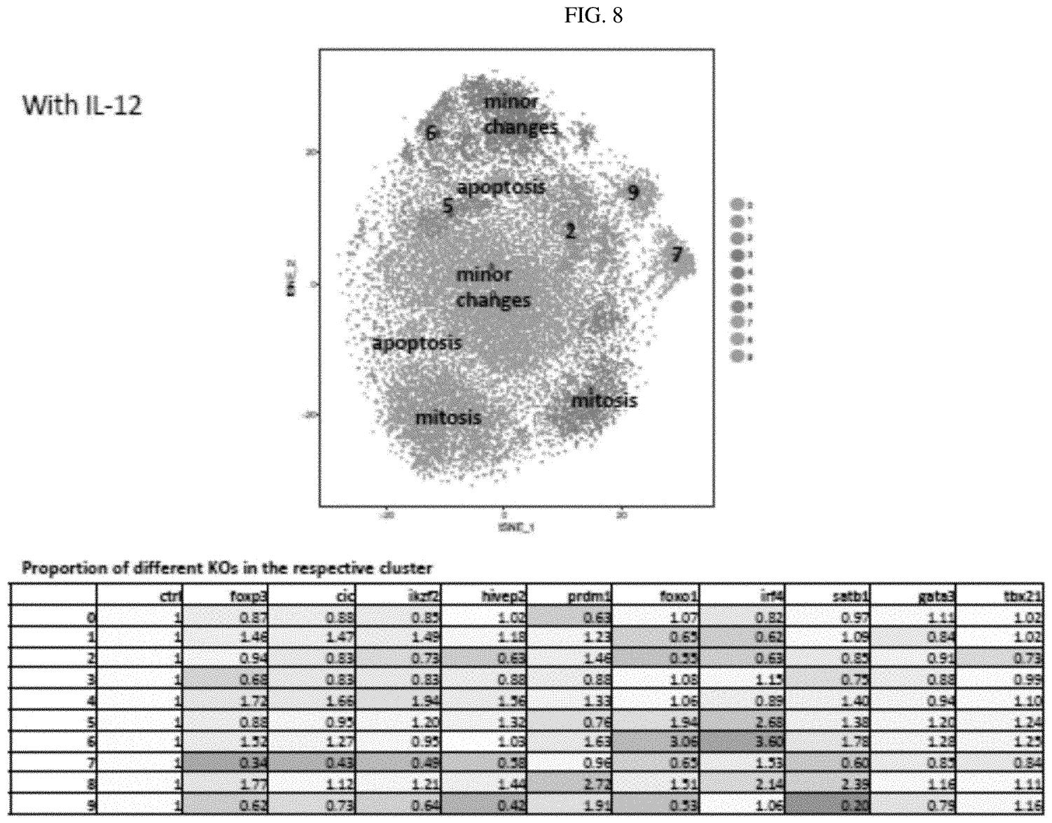

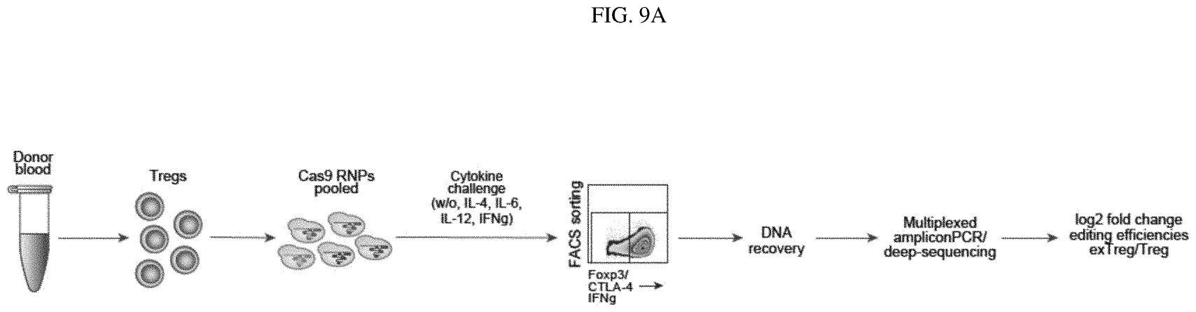

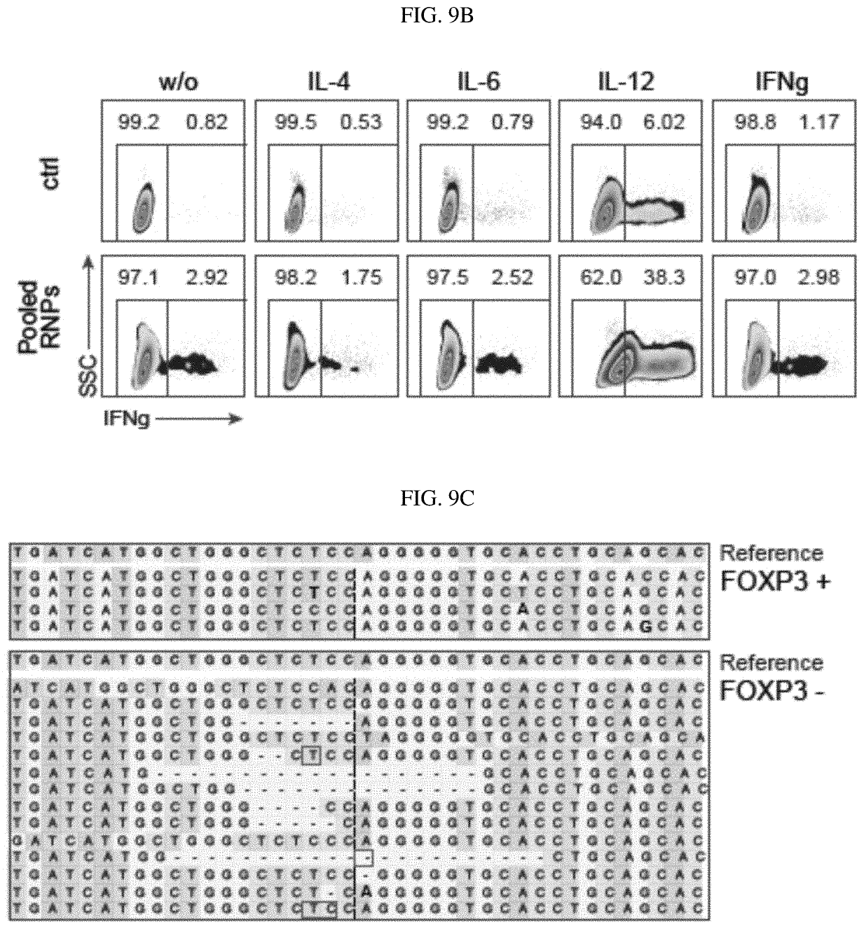

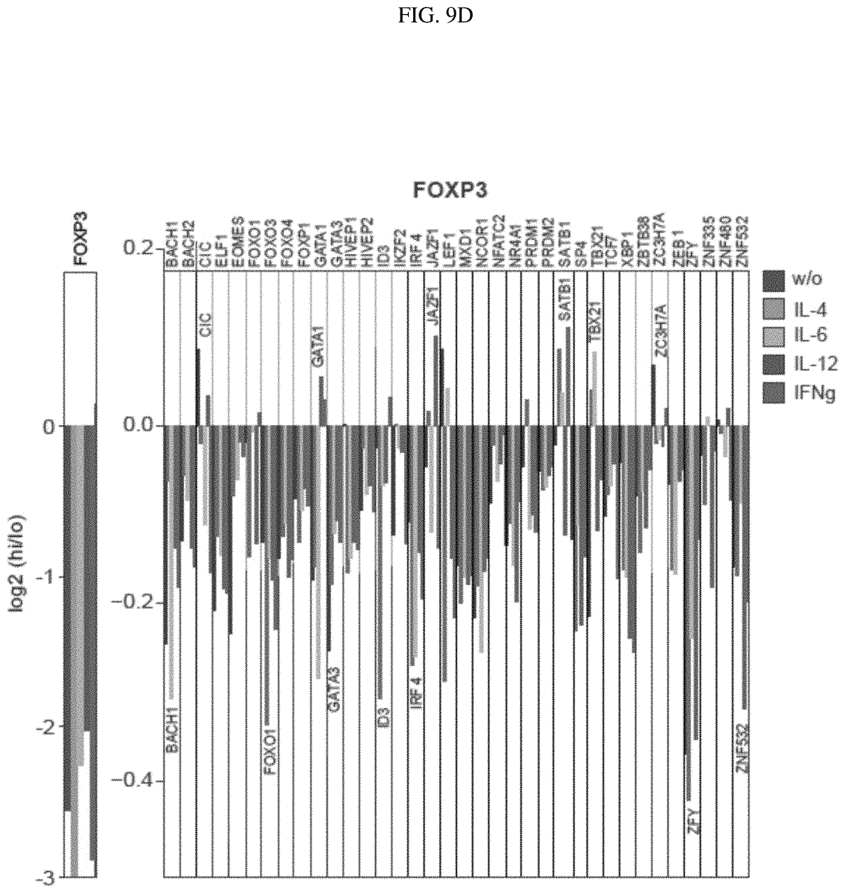

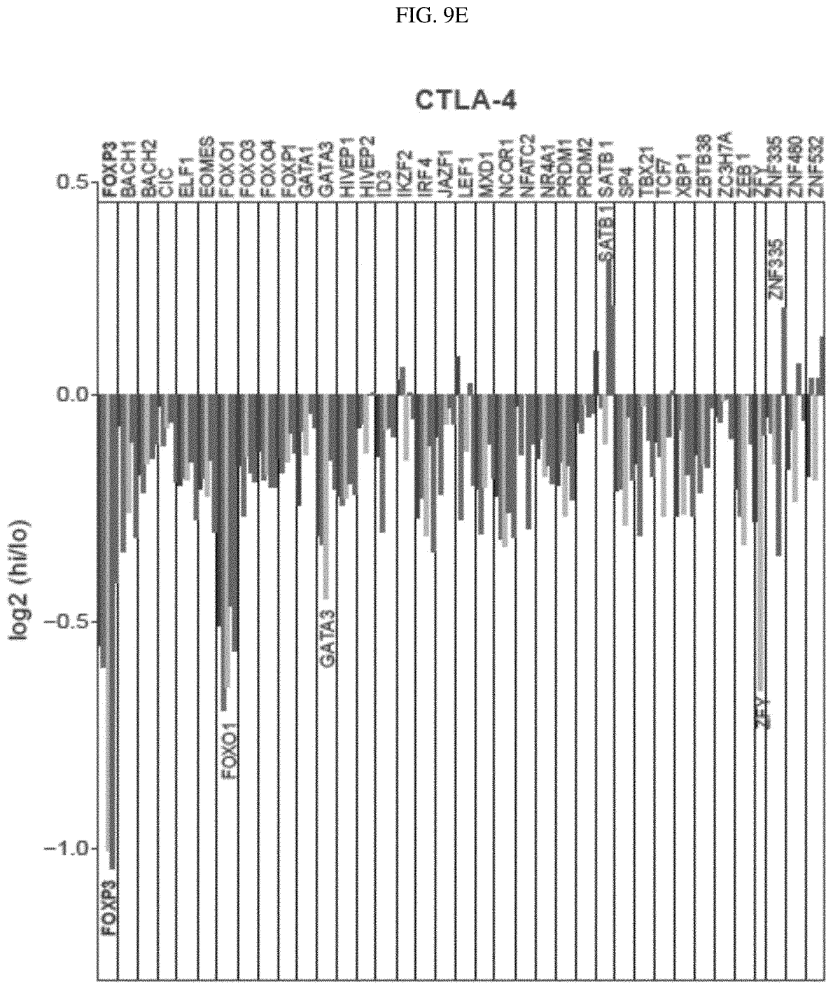

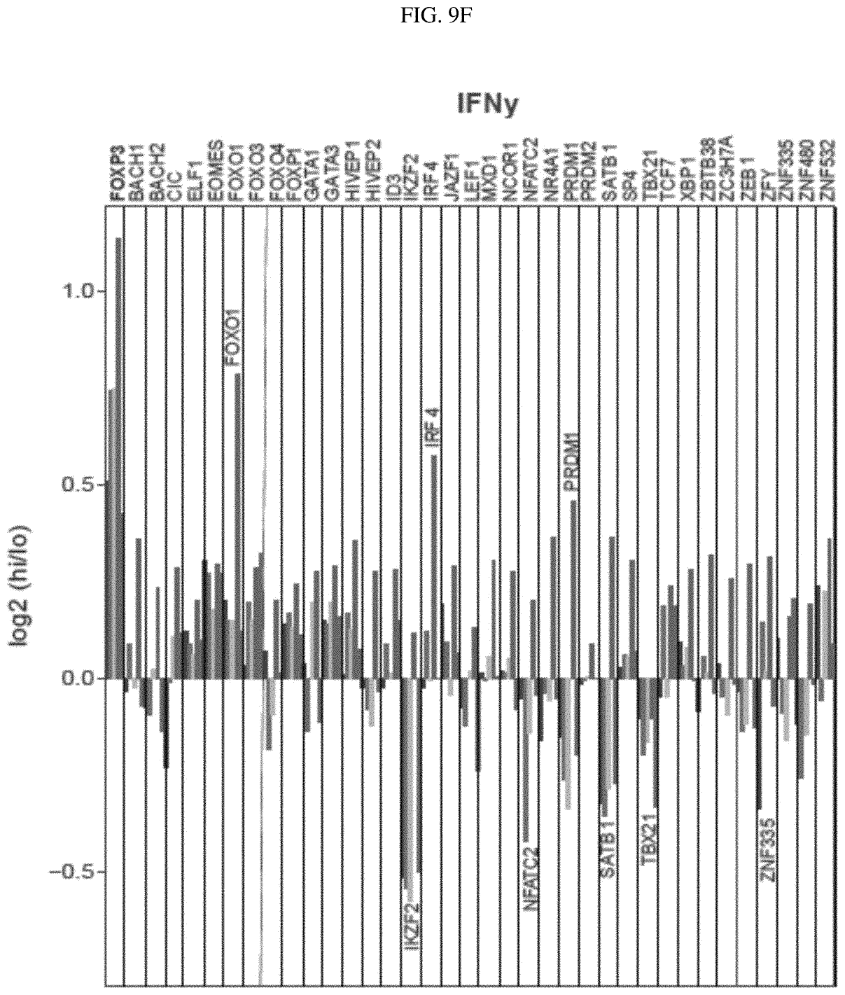

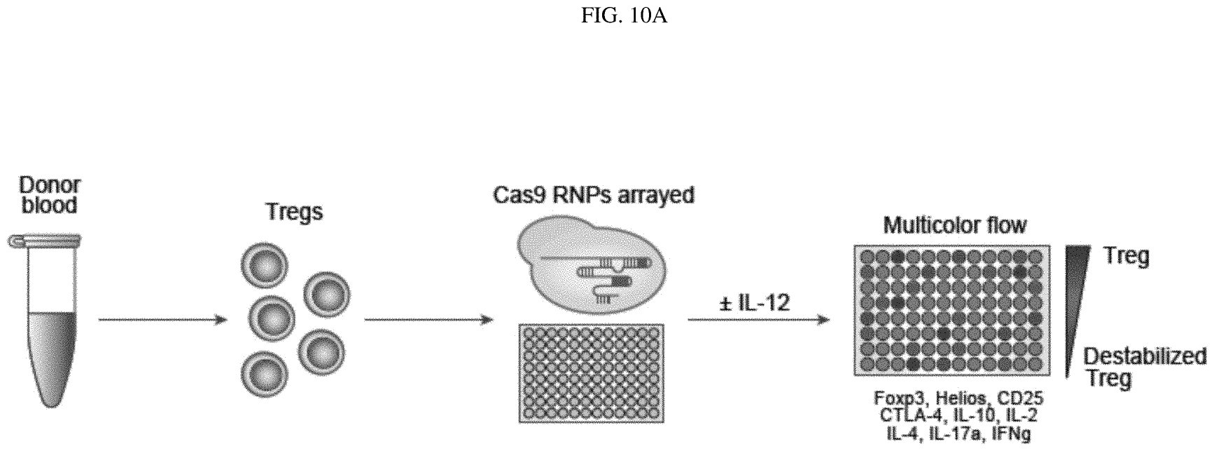

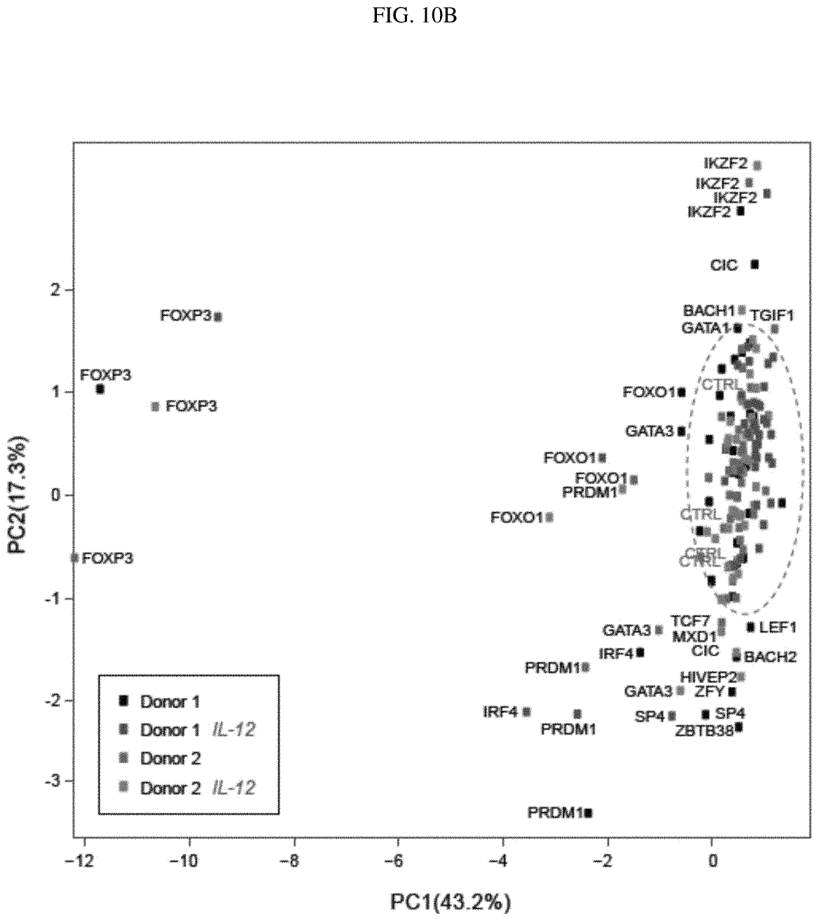

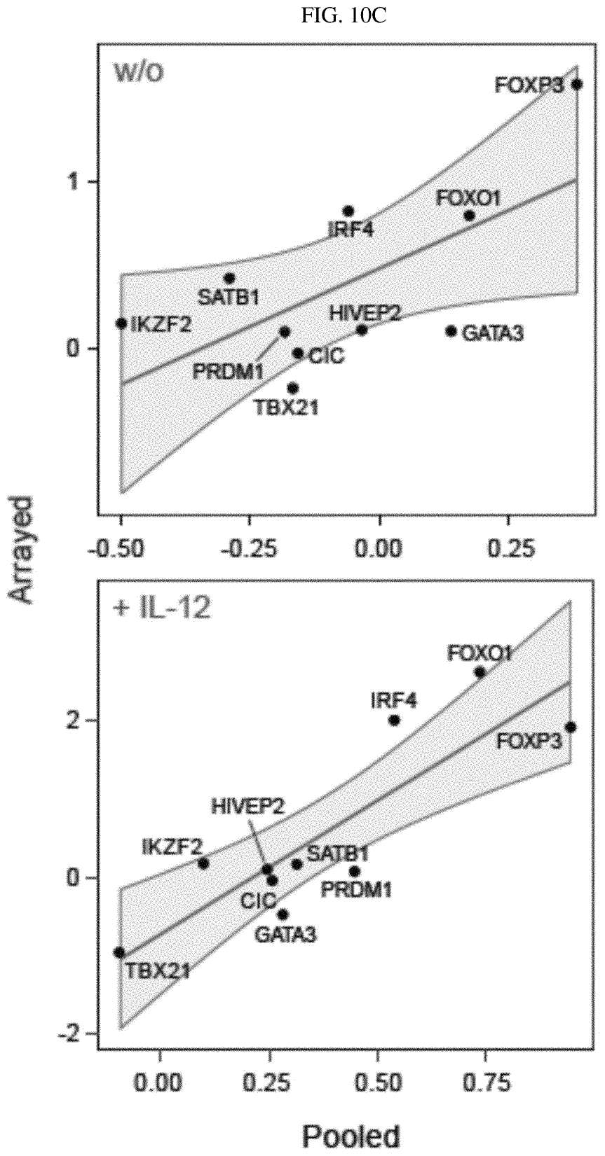

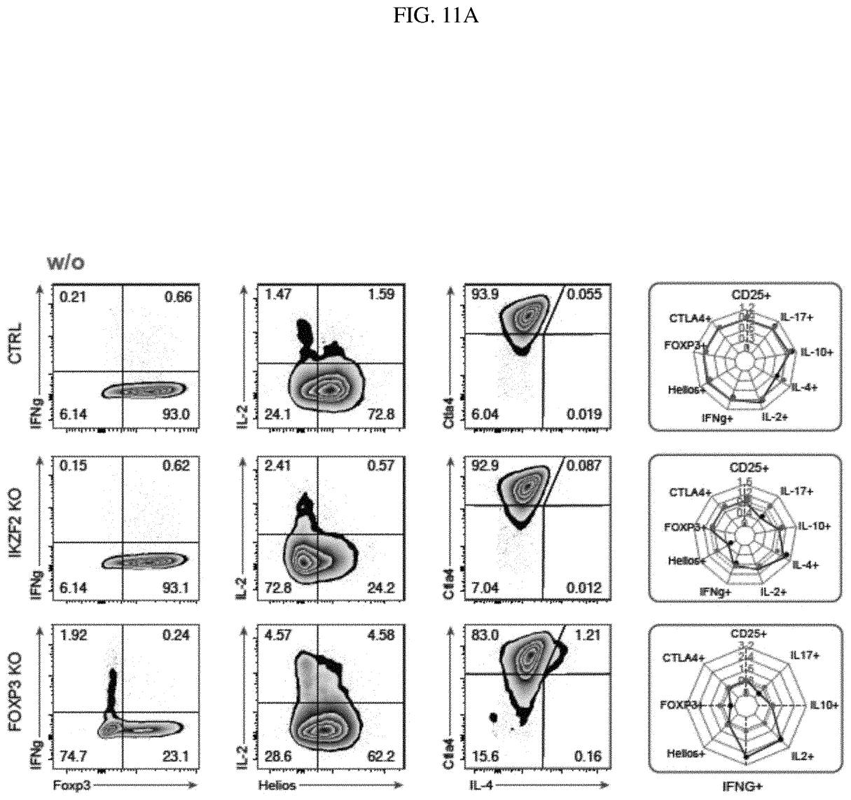

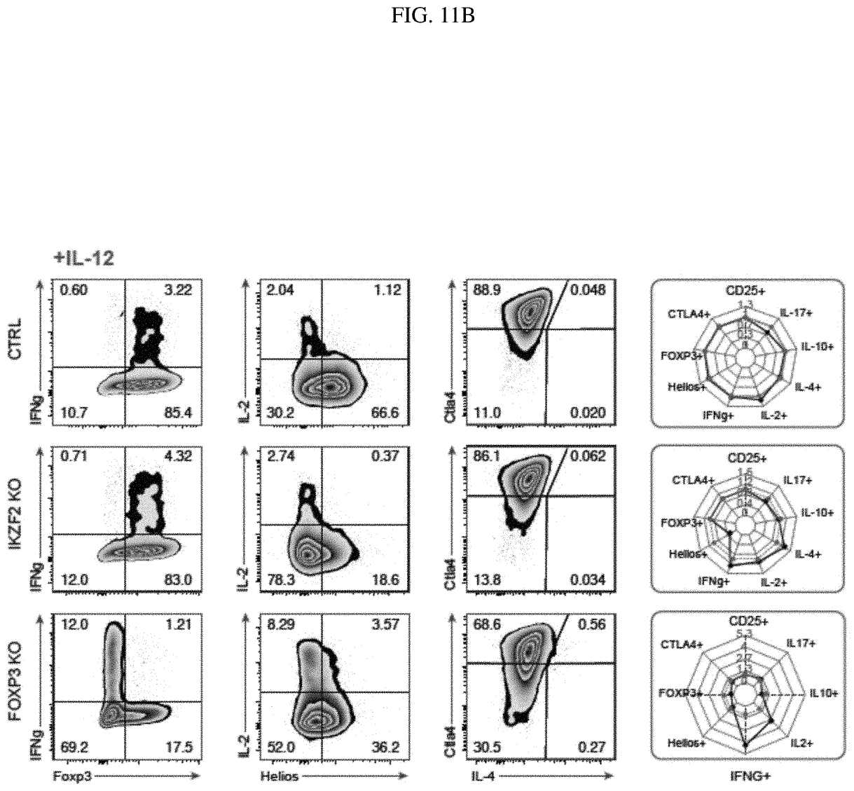

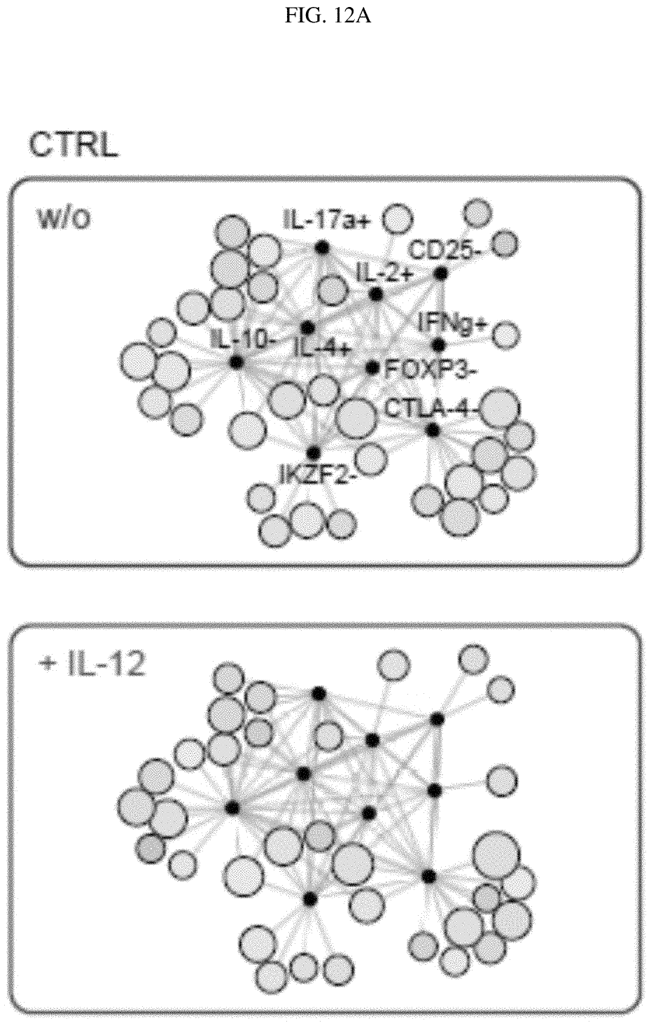

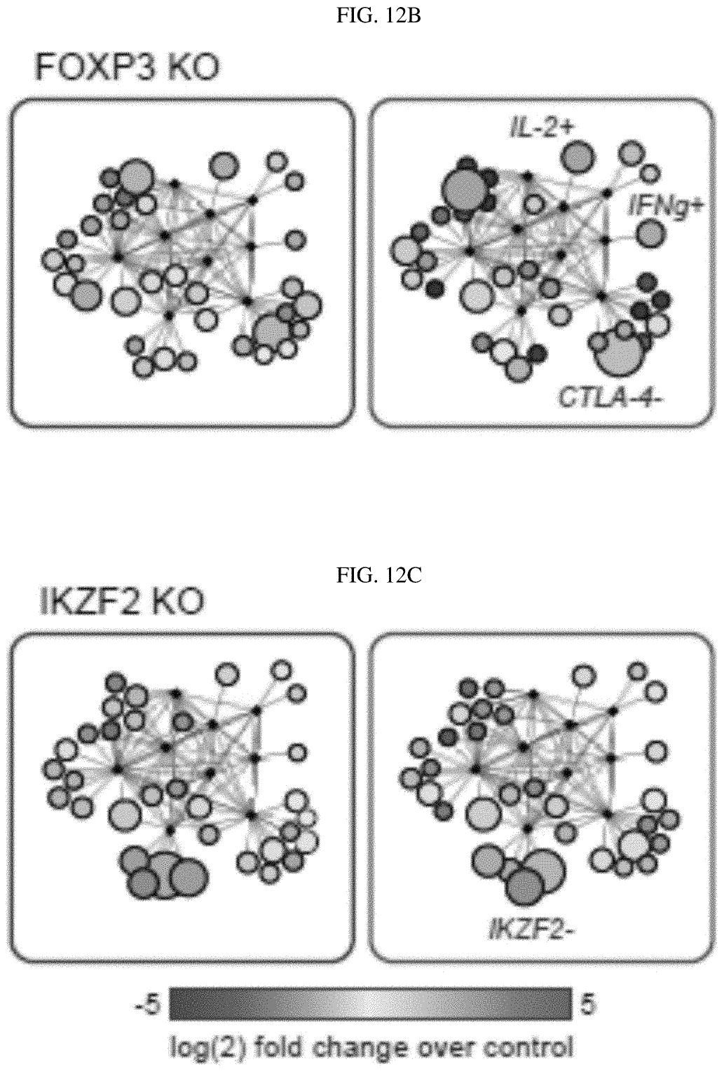

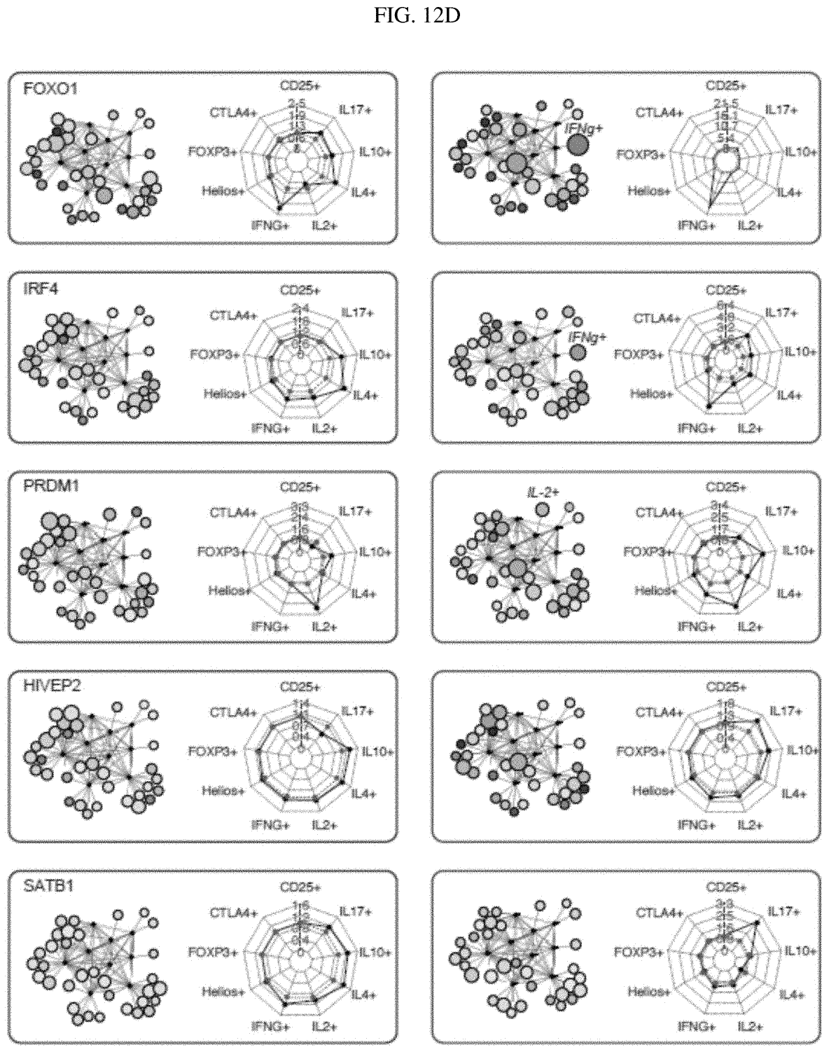

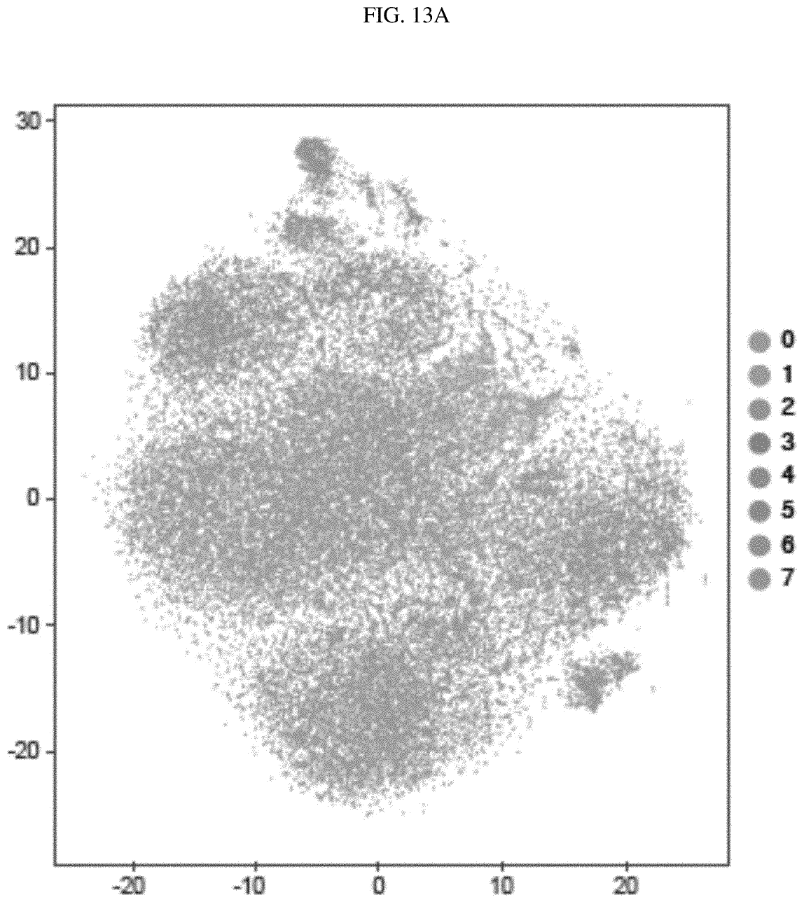

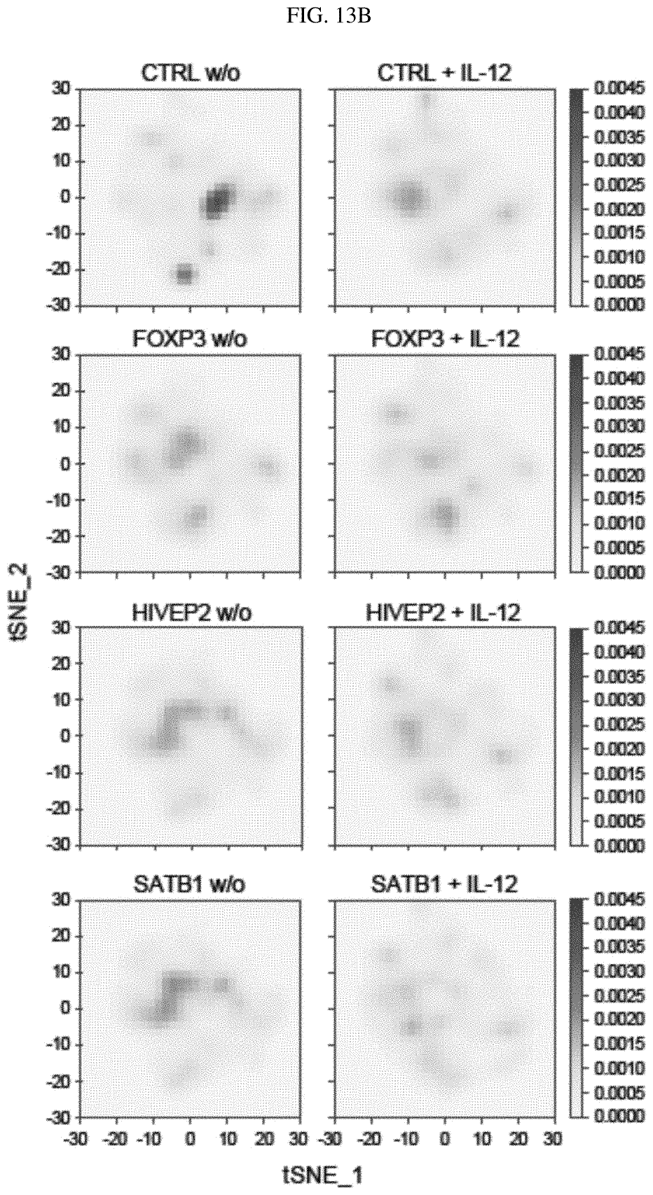

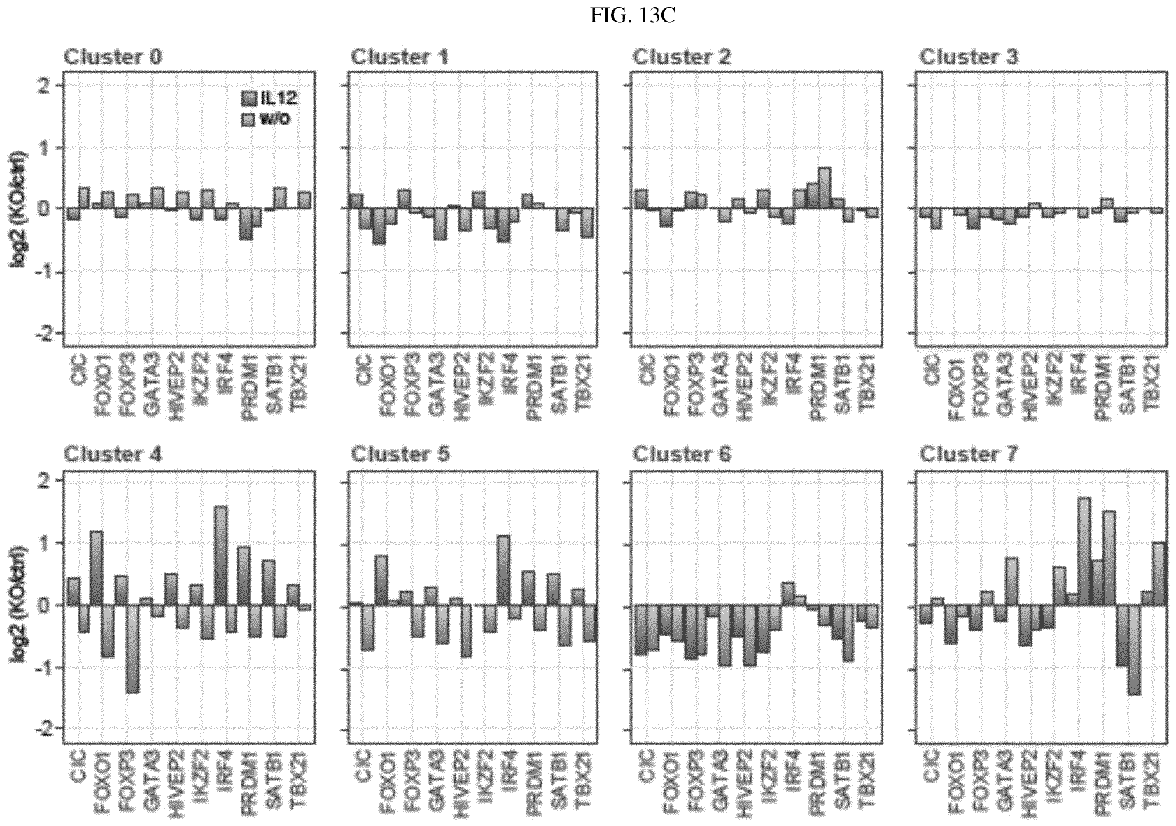

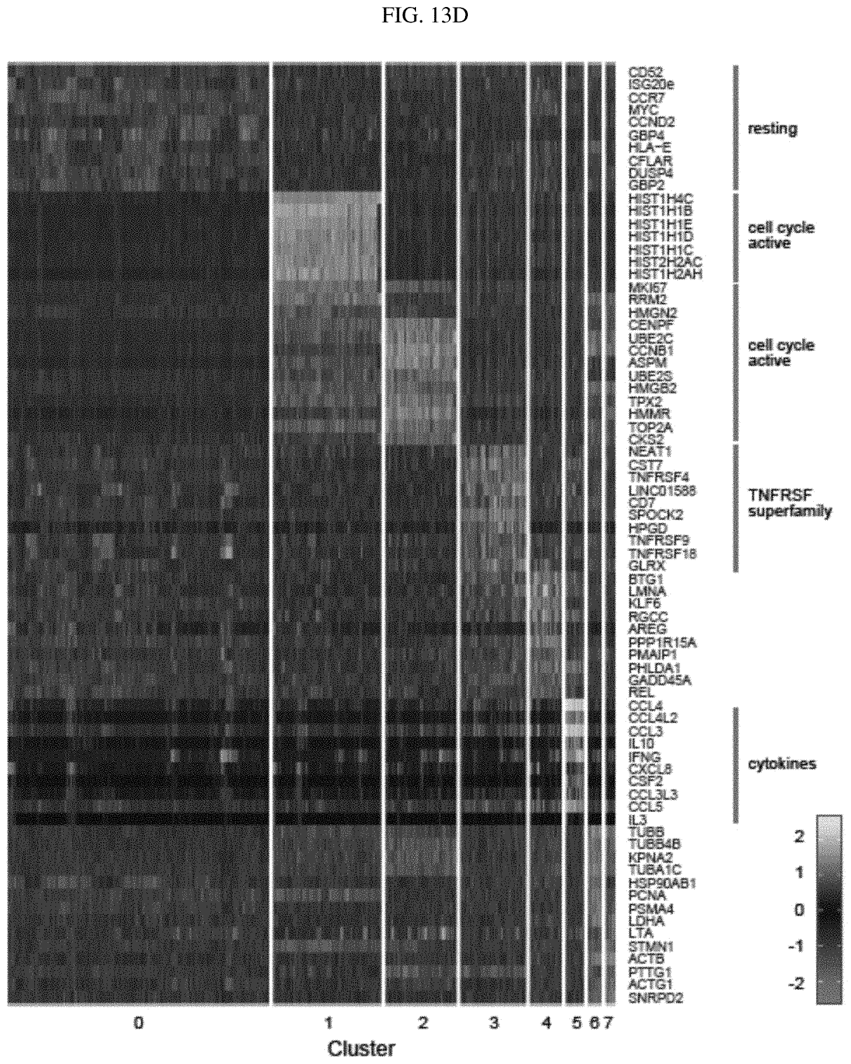

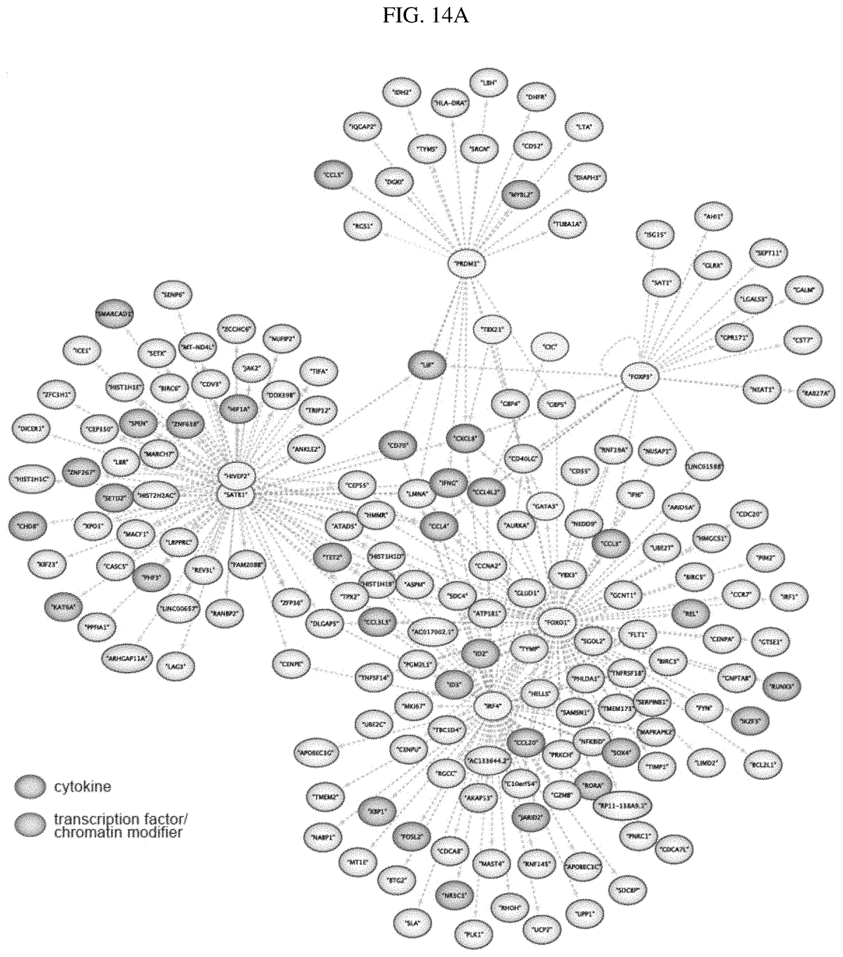

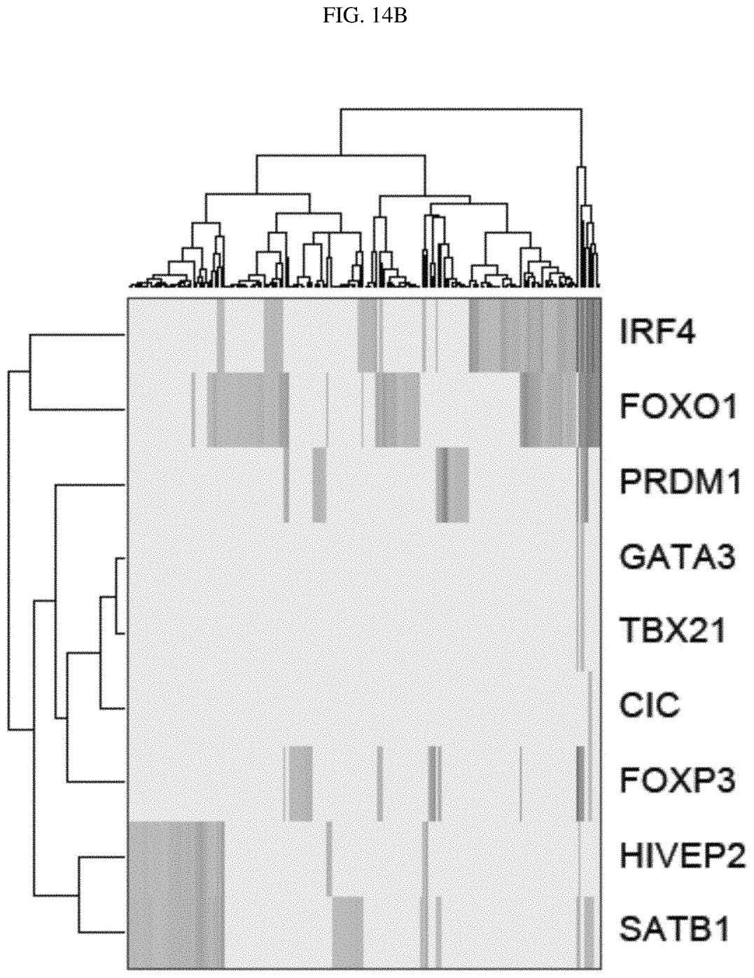

FIG. 8 shows clustering of single cell RNA-sequencing (scRNA-seq) data of 10 transcription factors. Nine different clusters could be identified. The table in FIG. 8 shows how each transcription factor was represented in the individual clusters compared to control cells (values normalized to control cells). Initial analysis assigned a function to the individual cluster, e.g., mitosis, apoptosis, etc. Clusters that showed different enrichment of knock-outs and were not connected to cell proliferation or cell death were focused on (e.g., clusters 2, 5, 6, 7, and 9).

### Example 11—Identification of Candidate TFs Regulating Treg Cell Identity

Regulation of canonical effector molecules is critical for Treg function. Treg suppressive mechanisms depend upon their ability to highly express CTLA-4 and FOXP3, as well as their ability to repress expression of pro-inflammatory cytokines including IFNg. It was sought to identify transcription factors that are essential for regulating these core Treg genes. 25 candidate TFs, including known Treg TFs FOXP3 and IKZF2 (Helios), were chosen based on preferential expression in Tregs compared to other CD4+ T cell subsets (Epinomics Roadmap; Farh & Marson et al., 2015). It was hypothesized that Treg-specific intragenic demethylated enhancer regions potentially mark core Treg TFs in addition to FOXP3 (Polansky et al., 2008); thus, 4 additional TF genes were also selected based on preferential demethylation of intragenic enhancer regions in human Tregs relative to conventional CD4+ T cells (Morikawa et al., 2014). TCF7, PRDM1, JAZF2 and HIVEP2 loci each contain at least two intragenic Treg demethylated regions. 11 additional TFs were chosen based on the described effects on murine T cell gene regulation—which includes known FOXP3 interaction partners like GATA3, FOXP1, IKZF2 and IRF4—or factors that control the cellular cytokine secretion patterns including FOXO1 and BACH2 (Rudra et al., 2012; Konopacki et al., 2019; Kwon et al., 2017; Ouyang et al., 2012; Roychoudhuri et al., 2013).

### Example 12—Pooled Cas9 RNP Screens Identify Transcriptional Regulators of Treg Cell Identity

To quickly dissect functional effects of the 40 candidate TFs on Treg cell identity, a pooled Cas9 RNP approach in ex vivo cultured primary human Tregs was developed. Each TF was targeted with a gRNA, selected based on predicted and experimentally validated on-target editing efficiency (details: Material and Methods). For each of the 40 genetic loci, one control gRNA targeting a neighboring site with no known function within 1 kb of the target was included. The gRNA pool was nucleofected into the human Tregs and incubated for 72 hours with IL-2 alone (“non-treated”) or in combination with IL-2 and different pro-inflammatory cytokines (IL-4, IL-6, IL-12 or IFNγ). Afterwards, cells were sorted based on their expression levels of Treg markers (FOXP3 and CTLA-4), or based on their expression levels of the pro-inflammatory effector cytokine IFNg (FIG. 1B).

As a final readout, it was assessed which Cas9 RNP-mediated mutations drive dysregulation of the assessed target in Tregs. It was reasoned that functional mutations would be preferentially enriched in cells with dysregulated target gene expression. The DNA of the sorted cell populations was subjected to multiplexed amplicon sequencing followed by NGS sequencing to determine the individual insertion and deletion (indel) mutation distributions. For 37 of the 40 loci, the indel frequency in the sorted cell fraction could be successfully determined (the IKZF4, TGIF1 and ZNF831 target loci did not amplify well with PCR). Indels in any of the amplicon control regions or in Tregs nucleofected with non-targeting control RNP could not be detected, showing the specificity of the approach and that no artefacts were generated during intracellular staining and DNA recovery (data not shown). The FOXP3 negative population had high frequency of large indels in the FOXP3 locus compared to the still FOXP3-expressing Tregs which showed only rare point mutations (FIG. 1C) proofing the functionality of this novel screening approach. Based on the determined indel frequencies, the log of the ratio of edit enrichment in the “marker-high” cells to the edit enrichment in the “marker-low” cells was calculated (FIGS. 1D-1F).

The first target analyzed was the Treg master TF FOXP3. As expected, direct targeting of the FOXP3 gene had the strongest effect on FOXP3 expression. Both known regulators of FOXP3 expression could be identified in the screen like IRF4 and GATA3 as well as novel factors. For example, deletion of BACH1 and ZNF335 reduced FOXP3 levels. Interestingly several TF KOs reduced FOXP3 expression in a strongly cytokine dependent manner, like ID3 and FOXO1 after IL-4 treatment. The majority of the TFs tested stabilize directly or indirectly FOXP3 expression. These very tight transcriptional regulation further emphasizes the fundamental importance of FOXP3 for Treg function. The ablation of a few TFs could however even increase or stabilize FOXP3 expression above regular levels. These upregulations were again strongly dependent on the cytokine stimulation conditions like for example in the case of CIC (non-treated, IL-12), GATA1 (IL-12) and SATB1 (IFNg, IL-4) (FIG. 1D). These conditions can give insights into the regulation of Foxp3 expression and how Tregs can potentially sustain their cell identity in specific chronic pro-inflammatory environments.

CTLA-4 is a Treg key effector protein and expression on Tregs essential for immune homeostasis. Similar effects as observed for FOXP3 could also be detected for CTLA-4. Most TF KO conditions tested reduced CTLA-4 expression levels showing the tight transcriptional control of this effector molecule. Only IFNg conditioned SATB1 and ZNF335 KO cells could even further increased CTLA-4 expression. Higher or more stable CTLA-4 expression has potential application in treatment of autoimmune diseases. CTLA-4 downregulation was most pronounced in the FOXP3 and FOXO1 KO conditions. The FOXO1 KO phenotype is consistent with previous studies in mouse models (Kerdiles et al., 2010). However, in FOXP3 KO mice the CTLA-4 expression levels on T cells are increased (Fontenot et al., 2003). This result further underlines the importance of analyzing transcriptional circuits in human Tregs.

IFNg has been described as a classical Th1 cytokine. However, IFNg secreting Th1-like Tregs have been characterized and are thought to be beneficial in the tumor microenvironment to support an efficient anti-tumor response. IFNg secretion can be strongly boosted by an IL-12-triggered positive feedback loop (Becskei et al., 2007). The largest induction of IFNg secretion could be detected in FOXP3 KO Tregs after IL-12 stimulation. Ablation of IRF4, PRDM1, or FOXO1 in combination with IL-12 conditioning also induced IFNg-producing Tregs very efficiently. In murine Tregs FOXO1 represses IFNg gene transcription (Ouyang et al., 2012); this mechanism seems to be conserved in mouse and human. A reduction of the already low IFNg-secretion in control Tregs was achieved by deleting TBX21 (T-bet) deletion, the key regulator of IFNg induction. IFNg secretion was only reduced in a few tested conditions including IKZF2 (Helios) and PRDM1 KO cells in all conditions besides IL-12 stimulation thereby stabilizing the suppressive Treg phenotype. This result contradicts observations in murine Helios-deficient Tregs which secrete pro-inflammatory cytokines including IFNg (Kim et al., 2015). However, Helios also acts early during Treg development, an effect which is not included in the system and could explain the discrepancy of the human and the murine results.

In summary, pooled Cas9 RNP screens allowed the quick identification of TFs regulating human Treg cell identity in various pro-inflammatory microenvironments based on indel frequencies in the respective genetic loci. FOXP3 is one of the key factors regulating Treg cell identity by adjusting expression levels of all three effector molecules tested. Also, IRF4 and FOXO1 are regulating all of these molecules, however it cannot be excluded that these effects are (partially) dependent on FOXP3 regulation by these factors indicating the need for further genetic dissection of these KO phenotypes. TF KO conditions that positively or negatively affect one or several effector molecules in several contexts can be detected, allowing the efficient test of potential cell engineering interventions. These phenotypes only partially overlap with described murine Treg phenotypes highlighting the importance to further genetically dissect human Tregs.

### Example 13—Arrayed Cas9 RNP Screen for in-Depth TF KO Treg Cell Phenotyping

To validated the findings of the pooled Cas9 RNP screen, the TF knockouts were repeated in an arrayed 96 well format. This allowed not only to confirm the observed effects of each gene perturbation, but also to further characterize the quantitative effects of each gene perturbation on a panel of core Treg proteins with flow cytometry. Each TF was targeted with 3 different gRNAs in Tregs from two human blood donors to reduce the risk of phenotypes attributable to off-target effects individual gRNAs or donor-specific effects. The consequences of each genetic perturbation were assessed in the presence and absence of IL-12, which had pronounced effects in the pooled Cas9 RNP screen (FIGS. 1B and 1D). Targeted cells were fluorescently stained for 9 canonical Treg and Teff protein markers (FOXP3, Helios, CTLA-4, CD25, IL-10, IL-2, IL-4, IL-17a, IFNg) and analyzed by high throughput flow cytometry to assess resulting changes in core Treg cellular identity (FIG. 2A). The PCA plot in FIG. 2B summarizes the results for all 9 markers in both donors for the 3 individual RNPs targeting each TF. The wells with non-targeting RNPs clustered together as did the majority of the conditions, consistent with modest effects of most perturbations. The FOXP3 and IKZF2 KOs separated from the other tested conditions, as expected due to known roles in Tregs and direct effects on proteins in the flow cytometry panel. Several TFs, for example IRF4, CIC and HIVEP2, after IL-12 treatment only led to a deregulation on one donor, indicating the need for further more detailed analysis. Targeting GATA3, PRDM1, and FOXO1 also caused distinct protein deregulation in both donors and both stimulation conditions, validating results observed in the pooled RNP screen. Overall, data in the arrayed gene KO experiments correlated well with those in pooled screens, especially the strong effects observed in the presence of IL-12 (FIG. 2C).

Next, the individual FACS markers for each TF KO were visualized to more granularly characterize which markers impact the loss of Treg cellular identity in the individual conditions. To assess the dominant effects of each TF, all flow cytometry results with personality plots that highlight the dominant effects of each KO (FIGS. 3A, 3B, and 4D) were summarized. For comparison two-dimensional FACS plots for some of the markers are depicted in FIGS. 3A and 3B. For example, IKFZ2 KO cells show in the steady state and IL-12 conditioning a reduction of IKZF2 and very minor changes in pro-inflammatory cytokine production. FOXP3 deletion resulted in a reduction of FOXP3 protein levels and an increased production of the inflammatory cytokines IL-4, IFNg and IL-2. The levels of IFNg and IL-2 can be further boosted by addition of IL-12. These results are highly comparable between different RNPs targeting the same TF and between different donors. However, what was challenging to determine with this analysis is whether a given KO generates one cell population with several deregulated markers, several cell populations defined by a single marker, or some combination of both.

### Example 14—Multidimensional Analysis of TF KO Tregs Reveals KO Sub-Phenotypes

It was hypothesized that the transcriptional signature following CRISPR-mediated TF KO is lost both gradually and stochastically. During this process, several sub-phenotypes could be potentially generated. To identify such sub-populations, multidimensional characterization of the data was performed that moved beyond two-dimensional FACS-plots. Therefore, a single-cell pipeline initially designed for CyTOF data (SCAFFOLD, Spitzer et al., 2015) was utilized to generate a comprehensive, multidimensional analysis.

Firstly, subpopulations in the two positive controls IKZF2 and FOXP3 KO Tregs were analyzed. As expected, IKZF2 KO samples showed a large reduction in Helios expression independent of stimulation (FIG. 4C). The FOXP3 KO phenotype depicted with SCAFFOLD is more complex, because the ablation of FOXP3 results in the de-regulation of several other markers (FIG. 4B). Addition of IL-12 to the FOXP3 KO cells boosted the deregulation of the individual clusters. These findings are consistent with previous analysis (FIGS. 3A and 3B). Based on SCAFFOLD analysis, the sum of all cluster changes for each TF KO/stimulation condition was calculated to rank each sample by the loss of Treg cell identity. The results between both donors are highly consistent. By integrated amplicon sequencing data (89% of amplicons sequenced for the IL-12 stimulating conditions; for each TF at least two amplicons were sequenced) 9 TFs with the strongest phenotypic Treg deregulation were chosen. TBX21 was included as a negative control for IL-12 stimulating condition. In FIG. 4F the SCAFFOLD plots of these 10 TFs are depicted alongside the corresponding personality plots.

Generally, in all conditions besides the negative control TBX21, IL-12 enhances the deregulation after KO indicated by overall changes in cluster size and phenotype. PRDM1 KO Tregs showed a very distinct loss of Treg cell identity, and is the only condition besides FOXP3 KO that exhibits an IL-2 secretion subpopulation. Interestingly, FOXO1 and IRF4 deletion led to similar patterns of deregulation, most notably with a defined IFNg secreting cluster. IL-12 treated CIC, HIVEP2 and SATB1 KO Tregs showed minor phenotypes in the pooled RNP screen and in the “traditional” FACS analysis (personality plots; FIG. 4F), but ranked high in the SCAFFOLD analysis indicating a complex pattern of marker deregulation. Potentially IRF4/FOXO1 and CIC/HIVEP2/SATB1 regulate similar genes or even gene networks resulting in a similar KO phenotype (FIG. 4D). In summary SCAFFOLD is an efficient analysis method to assess the phenotypic diversity of Treg deregulation following TF ablation.

### Example 15—Unbiased Analysis of CRISPR-Induced Treg Heterogeneity with scRNA-Seq

Characterization of 40 TFs with Cas9 RNPs and flow cytometry revealed a core set of TFs with critical control of key protein targets in the presence and absence of inflammatory cytokines. The next step was to map the full molecular programs controlled by each of 10 core TFs with or without IL-12 conditioning. Therefore, targeted knock out of each TF was coupled with scRNA-seq to assess cellular phenotypes arising from the loss each of these TFs in human Tregs.

A large resource of scRNA-seq in human Tregs with and with gene knock out was generated. 54,424 cells passed the quality control after sequencing and were included in the analysis. The most highly variable genes (618 genes) in this data set were identified and used the gene list as an input for dimensional reduction. Using this approach eight different Treg clusters in the pool of all cells could be identified (FIG. 5A). All of these clusters contained control Tregs, with higher amounts in cluster 0 and 1 (FIG. 5B) indicating an intrinsic heterogeneity in the expanded Tregs. After IL-12 incubation, the control cells are more evenly distributed between the different clusters, indicating that pro-inflammatory conditions may introduce a higher level of cellular diversity (FIG. 5B). Besides cytokine treatment TF ablation significantly changed the cellular distribution of the clusters. For example, the distribution of FOXP3, SATB1 and HIVEP2 KO cells with and without IL-12 treatment differed noticeably from the respective control samples (FIG. 5B). Interestingly, SATB1 and HIVEP2 show a very comparable cell distribution in non-treated as well as IL-12 conditioned KO cells, indicating a similar destabilization pattern as seen before in the SCAFFOLD analysis of the arrayed screen. The normalized frequencies of each KO/stimulation condition are summarized in FIG. 5C. Overall, these analyses indicate an increase in Treg cellular diversity following ablation of TFs and/or IL-12 conditioning.

Next, the main driving forces forming the separate clusters were identified. The gene list in FIG. 5D comprises the top 10 upregulated genes for each cluster identified by differential gene expression analysis. Clusters 0-2 are dominated by cell-cycle and cell survival genes (cluster 0: resting CCR7-high Tregs; cluster 1 and 2: cycling Tregs). Tregs are reduced after ablation of the tumor suppressor PRDM1 in cluster 0 and enriched in cluster 2. Tregs located to cluster 4 express the TF c-REL which is important for the maintenance of activated Tregs (Gindberg-Bleyer et al., 2017) as well as genes connected to apoptosis.

Cluster 3, 5, 6 and 7 are dominated by genes affecting Treg function. Tregs located to cluster 3 express the co-inhibitory receptors of the TNFR superfamily OX40 (TNFRSF4), 41BB (TNFRSF9) and GITR (TNFRSF18) on high levels rendering them to be less suppressive (REF). However, only small variations of TF KO representation in this cluster were observed, which seems to be therefore be an intrinsic population in healthy individuals.

Cells locating to cluster 5 express immunosuppressive IL-10 in parallel to various pro-inflammatory cytokines like IFNg and IL-4; this effect appears predominantly in FOXO1 and IRF4 KO cells after IL-12 treatment. The strong cytokine-secretion pattern in combination with IL-12 supports the idea that this cluster represents highly-activated Th1-like Treg cells.

Using a KEGG module over-representation test, it was determined that cluster 6 and 7 contain cells experiencing a metabolic shift towards gluconeogenesis and glycolysis. Regularly Tregs use the more energetically favorable oxidative phosphorylation pathway. Cluster 6 is enriched for IRF4 KO Tregs while cluster 7 is depleted of SATB1 KO Tregs and highly enriched for Tregs after IRF4 or PRDM1 ablation. In summary TF ablation and/or pro-inflammatory IL-12 stimulation can influence these parameters and thereby location to individual clusters. The main driving forces defining the different clusters are cell cycle/cell survival, co-inhibitory receptors, cytokine secretion patterns and cell metabolism.

### Example 16—Identification of Gene Modules Regulating Treg Cell Identity

To better understand the gene modules that are directly or indirectly regulated by individual TFs, force-directed network graphs that illustrate the relationships between TFs and genetic modules were constructed. They were focused on the IL-12-treated samples to better understand the action of genetic circuitry in the context of pro-inflammatory environments. The analysis focuses on gene modules regulated by FOXP3, IRF4, FOXO1, PRDM1, SATB1 and HIVEP2 after IL-12 treatment (other tested TFs show no or only minor gene module regulation). Interestingly, in the center of the graph are a set of proinflammatory cytokines LIF, CXCL8, IFNg, CCL4 and CCL4L2 as well as CD40LG. These molecules are repressed by several TFs in parallel indicating the importance of the absence of these proteins for regular Treg function.

FOXP3 regulates a unique gene module largely separate of the other TFs, supporting the unique role of FOXP3 in Treg function. FOXP3 also increases its own expression in a positive feedback loop. PRDM1 also controls a distinct module that barely overlaps with those of the other TFs. PRDM1 upregulates a few genes, including the G-protein regulator RGS1, which regulates Treg migration, but mainly acts as a transcriptional repressor.

IRF4 and FOXO1 regulate distinct genes in each case as displayed in FIGS. 6A and 6B. However, the IRF4 and FOXO1 gene networks show great overlap (FIGS. 6A and 6B). Gene suppression seems to be reinforced by both TFs in parallel, and the number of co-activated genes is comparably small. IRF4/FOXO1 control genes affecting cell proliferation and cell survival (Ki67, CCNA2, BTRC3). These TF also regulate, together or individually, a large number of TFs that have been described in regulating different aspects of Treg function like c-Rel, ID2, ID3, IKZF3, RUNX3 and XBP1 (Grindberg-Bleyer et al., 2017; Miyazaki et al., 2014; Klunker et al., 2009; Fu et al., 2012). The induced or repressed gene modules are highlighted in FIG. 6B.

SATB1 and HIVEP2 are another pair of TFs that co-regulate a large gene module showing even greater regulatory overlap than IRF4 and FOXO1. These two TFs co-activate a great number of genes including the TF HIF1alpha, which is involved in Treg differentiation and metabolism, and TET1, which is an enzyme regulating the general DNA hypomethylation patter in Tregs and especially the demethylation of FOXP3 CNS2—a hallmark of Treg stability (Yang et al., 2015) (FIG. 6A). Also in this cluster a multitude of TFs are getting directly or indirectly regulated.

SATB1 acts as a pioneer factor during Treg development (Kitagawa et al., 2017). In mature, murine Tregs loss of SATB1 expression results in reduced cell survival and suppressive function (Kondo et al., 2016). Interestingly, increased SATB1 levels also negatively affect Treg function, suggesting that SATB1 expression levels are strictly regulated in Tregs (Beyer et al., 2011). It is possible that the genome organizer SATB1 is highly regulated in order to fulfill its complex function, and that the novel Treg transcriptional regulator HIVEP2 may act in concert with SATB1 to “back up” or fine-tune this important regulator of Treg cell identity. HIVEP2 (also: Schnurri-2) has been described as negatively affecting selection during T cell thymic development. Besides that, Th1 and Th2 differentiation is affected in HIVEP2 KO mice and strongly biased towards Th2. However, the role of HIVEP2 on Treg function and stability has not been assessed before. A role of HIVEP2 as a regulator of Treg function acting in concert with SATB1 is described.

By integrating pooled and arrayed CRISPR screens with scRNA-seq, TFs crucial for Treg cell identity were identified and the corresponding gene networks under their transcriptional control were profiled. The analysis reveals that deregulation after TF ablation can result in diverse phenotypic subpopulations. Besides FOXP3, the 5 TFs PRDM1, FOXO1, IRF4, SATB1 and the novel Treg regulator HIVEP2 regulate large genetic modules that dictate cell cycle and metabolic states, as well as influence phenotypes marked by co-inhibitory receptors and distinct cytokine expression patterns.

As demonstrated in the Examples herein, it was analyzed—with pooled RNP screens, arrayed RNP screens, and scRNA-seq—the impacts of different TF deletions in human Tregs across various pro-inflammatory microenvironments: Th1-like (IL-12), Th-2-like (IL-4), Th17-like (IL-6), and cells conditioned with IFNg as it is relevant in the tumor microenvironment (Dominguez-Villar et al., 2011; Rivas et al., 2015; REF; Overacre-Delgoffe et al., 2017) to identify additional transcriptional key factors in human Treg function. By combining CRISPR-editing with scRNA-seq, distinct TF-specific genetic networks were elucidated. Key genetic modules of Treg function controlled by single TFs like FOXP3 and PRDM1 were identified. TFs that act in concert with each other were also identified: the co-repressors IRF4 and FOXO1 and the co-activators SATB1 and HIVEP2. With these methods, HIVEP2 was characterized as a novel regulator of Treg cell identity.

Pooled Cas9 RNP screens were established to quickly dissect gene function in primary cells. In pooled RNP screens, a mixture of RNPs targeting different genomic sides are nucleofected simultaneously into expanded Tregs, which allows the quick screen for various proteins affecting Treg function in various environments. Pooled RNP screens can be easily expanded to more target sites, generating a medium to high-throughput system. So far, all published pooled screening approaches in human T cells apply the introduction of gRNA libraries via lentivirus into the cells in combination with Cas9 protein or ribonucleoprotein nucleofection (Shifrut et al., 2018; Ting et al., 2018). These techniques allow genome wide screens, a range that is beyond the scoop of pooled RNP screens. However, in regular pooled CRISPR screens, the final readout is the frequency of a given gRNA in a selected cell population, which does not necessarily correlate with a successful edit. In pooled RNP screens, actual frame-shifting indels were used as final readout, allowing a very clean in-depth analysis of the nature of the indels. The results of the pooled RNP could be validated with arrayed RNP screens.

By integration of CRISPR KOs, cytokine stimulation, and scRNA-seq, genetic modules regulating different aspects of Treg function were identified. These gene modules are involved in multiple aspects of gene functions including regulation of TFs and histone modifiers, cell signaling, survival and the expression of key cell surface markers. FOXP3 and PRDM1 are regulating separate distinct gene module with minimal overlap to the other TFs. IRF4 and FOXO1 co-regulate—mainly co-repress—a multitude of different genes which include several TFs, cytokines, and co-inhibitory receptors. During Treg development, FOXO1 acts as a pioneer factor upstream of FOXP3. In FOXO1 KO mice, the development and function of thymic-derived Tregs—especially the expression levels of CTLA-4 and IFNg—are aberrant, a phenotype that is confirmed in human Tregs (Kerdiles et al., 2010). IRF4 has been described as crucial in Th2-like Treg development and in the function of mucosal Tregs (Zheng et al., 2009; Cretney et al., 2011). In these cells IRF4 is inducing PRDM1 and both TFs jointly control many key genes of Treg function (Cretney et al., 2011). Using the described approach, a distinct genetic module regulated by PRDM1 that shows basically no overlap with the IRF4/FOXO1 was identified. Furthermore, IRF4 is part of the “quintet” of TFs described to act in concert to lock in the FOXP3 signature together with FOXP1, ELF1, LEF1 and GATA3 (Fu et al., 2012). However, in the pooled and arrayed screening data, by using a distinct set of screening parameters, FOXP1, ELF1, and LEF1 ablation showed only minor phenotypic effects and the GATA3 KO phenotype was distinct from IRF4 KO.

The PBMC-derived human Tregs were expanded ex vivo before CRISPR editing. The KO Tregs were phenotyped 5 days after CRISPR editing. Perhaps more or slightly different KO phenotypes could have been detected at other time points. Furthermore, effects of TF KOs on Treg development, on tissue-specific Tregs, and long-term effects of TF ablation on Treg phenotypes were not addressed. Future studies will show if some of the detected KO phenotypes are Treg-specific or can also be detected in other T cell subsets.

The TFs SATB1 and HIVEP2 co-activate a multitude of genes including HIF1alpha, TET2, and several TFs. SATB1 is very strictly regulated and only expressed in low levels in Tregs (REF). On one hand, release of SATB1 out of this strict control results in the expression of pro-inflammatory effector cytokines (Beyer et al., 2011). On the other hand, SATB1 KO mice have significantly reduced Treg numbers (Kondo et al., 2016). HIVEP2 potentially adds another level of (co-)regulation to keep these gene levels in check. The amount of co-regulation is striking, leading to the question of whether HIVEP2 or SATB1 act in a protein complex together, or if SATB1 and HIVEP2 binding sites are often located in the same gene-regulatory elements. TFs often act in protein complexes which may control their activity and DNA recognition motif; further, IRF4, PRDM1, and SATB1 interactions and complex formation with other TFs or TF cofactors have been described (REF). Therefore, further studies are necessary to define the nature of the co-regulation in these modules.

Interestingly, haploinsufficient patients with HIVEP mutations resulting in no or non-functional protein have been described. These patients have developmental defects and intellectual disability (Srivastava et al., 2016; Steinfeld et al., 2016). However, no autoimmunity has been described in these patients so far. Presumably immune defects could only be detected in a homozygous knockout setting.

Genetic modules in ex vivo expanded mature human Tregs isolated out of peripheral blood, a cell source that is also used for Treg cell therapy approaches, were identified. These Tregs are highly stable, suppressive Tregs for the treatment of autoimmunity and potentially proinflammatory Tregs to enable an efficient anti-tumor response. Currently, polyclonal ex vivo expanded Tregs are in phase 1 and phase 2 clinical trials for the treatment of Type1 diabetes and organ rejection after transplantation (Bluestone et al., 2015; Chandran et al, 2017; Mathew et al., 2018). The results show the importance to genetically dissect human PBMC-derived Tregs to identify potential gene targets for therapy and that results generated in murine Tregs can only partially be transferred to the human setting.

Treg TF KO phenotypes showed a strong cytokine dependency. Several KO phenotypes only appear in combination with specific cytokine treatments like FOXP3 stabilization after FOXO3/IL-4 and GATA3/IL-12 ablation raising the idea that many repressed genes after TF KO can only be visualized in a challenge situation outside the steady state. So far, the knowledge around the driving cytokines at which disease stage is scarce. IL-17 seems to be a driver in multiple sclerosis one. In solid tumors IFNg-levels can shape the anti-tumor response. For future cell engineering approaches the microenvironment the engineered cells are exposed to can potentially play a crucial role.

As described herein, the characterization Treg gene networks can act as a starting point for Treg engineering approaches in the future. By understanding transcriptional regulation of Tregs safer Treg-based cell therapies can be created by choosing optimal gene targets, and selecting distinct Treg sub-populations. Looking forward, the growing understanding of how Tregs control their anti-inflammatory features and how they acquire pro-inflammatory functions could lead the way to the next generation of Treg-based therapies also in combination with cutting-edge CAR and TCR technologies.

### REFERENCES

1. Kang H M, Subramaniam M, Targ S, Nguyen M, Maliskova L, McCarthy E, Wan E, Wong S, Byrnes L, Lanata C M, Gate R E, Mostafavi S, Marson A, Zaitlen N, Criswell L A, Ye C J. Multiplexed droplet single-cell RNA-sequencing using natural genetic variation. Nat Biotechnol. (2018), 36(1):89-94.
2. Roth T L et al. Reprogramming human T cell function and specificity with non-viral genome targeting. Nature, accepted.
3. Rupp L J, Schumann K, Roybal K T, Gate R E, Ye J Y, Lim W A, Marson A. CRISPR/Cas9-mediated PD-1 disruption enhances anti-tumor efficacy of human chimeric antigen receptor T cells. Sci Rep. (2017), 7(1):737.
4. Hultquist J F*, Schumann K*, Woo J M, Manganaro L, Doudna J A, Simon V, Krogan N J, Marson A. A Cas9 ribonucleoprotein platform for functional genetic studies of HIV-Host interactions in primary human T Cells. Cell Reports (2016) 17(5):1438-1452.
5. Schumann K*, Lin S*, Boyer E, Simeonov D R, Subramaniam M, Gate R E, Haliburton G D E, Ye C J, Bluestone J A, Doudna J A, Marson A. Generation of knock-in primary human T cells using Cas9 ribonucleoproteins. PNAS (2015), 112(33):10437-42.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of modifying regulatory T (Treg) cell stability, the method comprising:
inhibiting expression of one or more transcription factors selected from the group consisting of CIC, FOXO1, FOXP3, IKZF2, PRDM1, HIVEP2, SATB1, IRF4, T-BET, and GATA3 and/or inhibiting expression of one or more gene products regulated by one or more of the transcription factors in the Treg cell.

2. The method of claim 1, wherein the inhibiting the expression destabilizes the Treg cell.

3. The method of claim 1, wherein the inhibiting the expression stabilizes the Treg cell.

4-8. (canceled)

9. The method of claim 1, wherein the inhibiting comprises reducing expression of the transcription factor, reducing expression of a polynucleotide encoding the transcription factor, or reducing expression of the gene products regulated by the transcription factor.

10. The method of claim 9, wherein the inhibiting comprises contacting a polynucleotide encoding the transcription factor or a polynucleotide encoding the gene products regulated by the transcription factor with a target nuclease, a guide RNA (gRNA), an siRNA, an antisense RNA, microRNA (miRNA), or short hairpin RNA (shRNA).

11. The method of claim 9, wherein the inhibiting comprises contacting a polynucleotide encoding the transcription factor or a polynucleotide encoding the gene products regulated by the transcription factor with at least one gRNA, wherein the at least one gRNA comprise a sequence selected from Table 2.

12. The method of claim 1, wherein the inhibiting comprises mutating a polynucleotide encoding the transcription factor or a polynucleotide encoding the gene products regulated by the transcription factor.

13. The method of claim 12, wherein the inhibiting comprises contacting the polynucleotide with a targeted nuclease.

14. (canceled)

15. The method of claim 13, wherein the targeted nuclease is an RNA-guided nuclease.

16-17. (canceled)

18. The method of claim 13, wherein the inhibiting comprises performing clustered regularly interspaced short palindromic repeats (CRISPR)/Cas genome editing.

19. The method of claim 1, wherein the Treg cell is administered to a human following the inhibiting.

20. The method of claim 1, wherein the Treg cell is obtained from a human, the Treg cell so obtained is treated to inhibit the expression, and then the cell having inhibited expression is reintroduced to the human.

21. The method of claim 20, wherein the inhibiting the expression results in the Treg cell with increased stability.

22. The method of claim 21, wherein the human has an autoimmune disease.

23. The method of claim 20, wherein the inhibiting the expression results in the Treg cell with decreased stability.

24. The method of claim 23, wherein the human has cancer.

25. A Treg cell made by the method of claim 1.

26. A regulatory T (Treg) cell comprising a genetic modification or heterologous polynucleotide that inhibits expression of one or more transcription factors selected from the group consisting of CIC, FOXO1, FOXP3, IKZF2, PRDM1, HIVEP2, SATB1, IRF4, T-bet, and GATA3 and/or inhibits expression of one or more gene products regulated by one or more of the transcription factors in the Treg cell.

27-29. (canceled)

30. A regulatory T (Treg) cell comprising at least one guide RNA (gRNA) comprising a sequence selected from Table 2.

31. The Treg cell of claim 30, wherein the expression of one or more transcription factors in the Treg cell selected from the group consisting of CIC, FOXO1, FOXP3, IKZF2, PRDM1, HIVEP2, SATB1, IRF4, T-bet, and GATA3 and/or the expression of one or more gene products regulated by the one or more of the transcription factors is reduced in the Treg cell relative to the expression of the transcription factor or gene product in a Treg cell not comprising a gRNA.

32-34. (canceled)
